(19) United States
(12) Patent Application Publication
(10) Pub. No.: US 2025/0385000 A1
(43) Pub. Date: Dec. 18, 2025

(54) DEVICE FOR MEASURING INFANT FEEDING PERFORMANCE

(71) Applicant: NEONEUR, Pennington, NJ (US)

(72) Inventors: Tom O'DONNELL, Philadelphia, PA (US); Daniel K. HEDGES, Owings Mills, MD (US); Jay N. ZEMEL, Elkins Park, PA (US); Caroline HOEDEMAKER, Pennington, NJ (US)

(73) Assignee: NEONEUR, Pennington, NJ (US)

(21) Appl. No.: 18/877,392

(22) PCT Filed: Jun. 21, 2022
(86) PCT No.: PCT/US22/34215

§ 371 Date: Dec. 20, 2024

(51) Int. Cl. G16H 50/20; A61B 5/00; G16H 20/60; G16H 40/20; G16H 40/67; G16H 80/00

(52) U.S. Cl. CPC G16H 50/20; A61B 5/4205; A61B 5/7267; A61B 5/742; G16H 20/60; G16H 40/20; G16H 40/67; G16H 80/00; A61B 2503/045; A61B 2562/0247

(57) ABSTRACT

A device for measuring or evaluating infant feeding performance is described herein. Specifically, the device improves the care of infants, particularly, high-risk infants by safely and effectively capturing quantitative infant feeding parameters, longitudinally incorporating this data into a database, and providing caregivers the means to optimize care, discharge protocols, optimize a length of stay, and monitor the infants remotely.

### FIELD OF THE EMBODIMENTS

The field of the invention and its embodiments relate to a device for measuring infant feeding performance, respiration, recovery, and neurological development for use in improving care of an infant.

### BACKGROUND OF THE EMBODIMENTS

Feeding is a complex task that requires an intricate coordination of sucking, swallowing and breathing. Sucking/feeding behavior is a sympathetic nervous system measure and early sucking patterns are considered the most purposeful motor skill of the newborn. (Craig, 1999). The initial ability to successful coordinate sucking, swallowing, and breathing develops between 34-36 weeks of gestation, very late in fetal development. Full term is considered 38-40 weeks of gestation. Moreover, researchers have demonstrated that early sucking performance is predictive of later neurodevelopmental outcomes. (Wolkhuis-Stigter, 2016). (Hiramoto, 2014). (Slattery, 2012).

A sizable number of preterm infants in the neonatal intensive care unit or NICU struggle with feeding due to poor respiratory control. (Law-Morstatt, 2003). The complexity of the task is increased when an infant struggles with respiratory coordination, which is often seen in prematurity. Respiratory dysregulation can result in an increased risk for feeding difficulties, and general health. Other factors which contribute to the lack of coordination include difficulties in exchanging oxygen efficiently, as in chronic lung disease of the preterm in suppressing breathing while sucking. (Law-Morstatt, 2003). Due to general immaturity, ventilation is sacrificed to the demands of feeding—the control of breathing seems to lag behind the reflexes driving sucking and swallowing. (Miller, 2004).

Sensorimotor control of oral feeding involves multiple central pattern generators to coordinate the suck-swallow-respirations and the integration and coordination of all three rhythmic motor behaviors. (Barlow, 2009). Breathing efforts appears to be the last function integrated into a successful feeding, making it important to accurately measure respiratory efforts.

Respiratory rhythms during feeding undergo developmental changes concurrent with maturation. (Gewolb, 2006). In fact, maturation of the breathing process is a continuous process that bridges fetal and neonatal life. (Abu-Shaweesh, 2004). Neonatal respiratory activity is characterized by irregularity and spontaneous changes. Preterm infants are less likely to initiate breathing and tend to have respiratory pauses. Specifically, respiratory rates in premature infants ranges from 40-60 breathes per minutes and preterm infants are prone to periods of apnea. (Pickler, 2006). The mechanism behind this immaturity of breathing responses is not clear, but seem to originate from the predominance of inhibitory input on respiratory centers. A concern for prolonged apnea has been suggested as an important cause of central nervous system damage in preterm infants. (Daily, 1968).

Likewise, the concern for tachypnea, or abnormal rapid breathing, is of equal concern as the infant is more likely to aspirate. Monitoring respiratory pattern in preterm infants becomes an essential part of safe clinical care since respiratory dysfunction will decrease an infant's ability to receive the necessary nutrients for growth. Monitoring an infant's respiratory rate provides objective data about an infant's ability to maintain oral feeding or revert back to tube assisted feedings. No tool exists to measure the coordination needed for safe oral feeding. No tool exists that can measure oral cavity changes in aggregate, resulting in sucking and swallowing, and separately for respiration of an infant. Measuring respiration of an infant externally, at a frequency faster than 1/seconds is a complexity managed herein. Thus, what is needed is a device capable of accurately measuring an infant's feeding performance.

### Examples of Related Art Include

U.S. Pat. No. 8,986,229 B2 describes an apparatus for evaluating the tongue strength of a subject during a sucking event that includes an insert positioned within a nipple element to provide an output in response to deformation of the nipple element during a sucking event. The output is at least one of resistive force exerted against the subject's tongue and movement measurement of deformation force exerted on the nipple element during the sucking event. The apparatus is configurable to evaluate nutritive sucking (NS) or non-nutritive sucking (NNS) capabilities of the subject. The insert may be a sensing device, a compliance element, an intermediate device or a combination of these. A coupling device is configured to position the insert relative to the nipple element and/or to receive output from the insert. A method includes evaluating tongue strength of a subject during NS or NNS and using inserts providing increasing levels of resistive force to exercise the subject's tongue.

U.S. Pat. No. 8,473,219 B2 describes a computational method for generating a feeding score for an individual infant based upon a comparison of feeding factor measurements obtained from the individual infant, values associated with the feeding factor measurements, and feeding parameter metrics from a population of infants having a similar gestational age as the individual infant.

U.S. Pat. No. 8,413,502 B2 describes devices, systems and methods for measuring infant feeding performance. The device includes a body portion, a pressure sensor and an integrated circuit. The body portion includes a first end for receiving a fluid, a second end mateable with a feeding nipple, and a conduit in fluid communication with the first and second ends. The pressure sensor is disposed in the body portion, is in contact with the fluid in the conduit, and generates a signal representing a pressure of the fluid passing through the conduit during a feeding session. The integrated circuit is disposed in the body portion and is electrically connected to the pressure sensor. The integrated circuit receives the pressure signal and determines a feeding factor over the feeding session indicative of the infant feeding performance.

Some similar systems exist in the art. However, their means of operation are substantially different from the present disclosure, as the other inventions fail to solve all the problems taught by the present disclosure.

### SUMMARY OF THE EMBODIMENTS

The present invention and its embodiments relates to a device for measuring or evaluating infant feeding performance. Specifically, the present invention and its embodiments relates to a device that improves the care of infants by safely and effectively capturing quantitative feeding parameters of infants, longitudinally incorporating this data into a database, and providing caregivers the means to optimize discharge protocols, optimize a length of stay, and monitor the infants remotely. This is particularly of interest for high-risk infants.

A first embodiment of the present invention describes a method executed by an engine on a computing device for quantitatively measuring infant feeding performance. The engine includes at least one artificial intelligence algorithm or machine learning algorithm. The method includes numerous process steps, such as: analyzing data received, in real-time, from a device affixed to a bottle and associated with a feeding instance of an infant during a first time period and receiving, from a user and via a graphical user interface (GUI) of the computing device, non-sensor data (e.g., identification data associated with the infant) during the first time period. The method also includes determining a baseline pressure from the data received from the device and adapting the baseline pressure to an actual reading.

Further, the method includes detecting sucks by comparing pressure fluctuations to known characteristics and detecting swallows by comparing pressure fluctuations to known characteristics. In some instances, the method may further include: detecting respiration by comparing temperature fluctuations to known characteristics. The method also includes calculating a second time period between the sucks and the swallows to detect bursts, calculating a third time period without the sucks and without the swallows, calculating at least one biomarker for the infant or the feeding instance, and outputting results of the feeding instance via the GUI to the user. Further, in some examples, the method may include displaying the results of the analysis in a telehealth interface executed on the computing device.

The results comprise curves depicting at least one of feeding trace data, maturation progress data, duration of quality data, and recovery data. The feeding trace data comprises at least one of: an average number of sucks/bursts in the first time period, an average length of time between bursts in the first time period, a frequency of sucks in a first two minutes in the first time period, a normal respiratory rate during the first time period, and an overall duration of feed during the first time period. In some examples, the method further includes utilizing the maturation progress data for curve tracking, slope tracking, and to determine a speed of change. The duration of quality data comprises a score for a quality of the feeding instance during a first two minutes of the feeding instance and a length of time the quality is maintained.

In some examples, the method further includes: detecting characteristic shapes of a specific patient population, a medical condition, or a given biomarker. In additional examples, the method includes: utilizing the at least one biomarker to calculate tracking indicators and comparing the at least one biomarker and the tracking indicators for the infant to determine how they change over time. In other examples, the method further includes: utilizing the at least one biomarker to calculate tracking indicators and comparing the at least one biomarker and the tracking indicators of the infant to a dataset to determine a normative comparison within a time derivative.

In some examples, the method further includes: receiving another set of data from a third-party and incorporating the other set of data into the analysis of the infant feeding instance. The other set of data comprises at least one of: a component of an oral feeding evaluation, a clinician evaluation of feeding quality, and an anthropometric evaluation completed prior to discharge and post-discharge.

A second embodiment of the present invention describes a computer system. The computer system includes: one or more processors, one or more memories, and one or more computer-readable hardware storage devices. The one or more computer-readable hardware storage devices contain program code executable by the one or more processors via the one or more memories to implement a method for quantitatively measuring infant feeding performance.

The method includes numerous process steps, such as: analyzing data received from a device affixed to a bottle and associated with a feeding instance of an infant during a first time period and receiving, from a user and via a graphical user interface (GUI), non-sensor data during the first time period. The method also includes determining a baseline pressure from the data received from the device and adapting the baseline pressure to an actual reading. Further, the method includes: detecting sucks by comparing pressure fluctuations to known characteristics, detecting swallows by comparing pressure fluctuations to known characteristics, detecting respiration by comparing temperature fluctuations to known characteristics, calculating a second time period between the sucks and the swallows to detect bursts, calculating a third time period without the sucks and without the swallows, and calculating at least one biomarker for the infant or the feeding instance.

Moreover, the method includes: outputting results of the feeding instance via the GUI to the user. The results include at least one of a developmental status of the infant, a feeding status quality for the feeding instance, a feeding status quality comparable during the feeding instance, and wherein the results comprise curves depicting at least one of feeding trace data, maturation progress data, and duration of quality data. The feeding trace data comprises at least one of: an average number of sucks/bursts in the first time period, an average length of time between bursts in the first time period, a frequency of sucks in a first two minutes in the first time period, a normal respiratory rate during the first time period, and an overall duration of feed during the first time period. The duration of quality data comprises a score for a quality of the feeding performance during a first two minutes of the feeding performance and a length of time the quality is maintained.

A third embodiment of the present invention describes a method executed by an engine on a computing device for quantitatively measuring infant feeding performance. The method includes numerous process steps, such as: analyzing data received from a device affixed to a bottle and associated with a feeding instance of an infant during a first time period and receiving, from a user and via a graphical user interface (GUI), non-sensor data during the first time period. Further, the method includes determining a baseline pressure from the data received from the device and adapting the baseline pressure to an actual reading. Next, the method includes: detecting respiration by comparing temperature fluctuations to known characteristics, calculating at least one biomarker for the infant or the feeding instance, and outputting results of the feeding instance via the GUI to the user.

It should be appreciated that a respiratory measurement component is incorporated into the device. Further, the respiratory measurement component fails to come into physical contact with the infant.

A fourth embodiment of the present invention describes a device affixed between a nipple and a bottle. The device includes embedded electronic components. The embedded electronic components include at least one sensor and a computerized data processing system. The at least one sensor is configured to capture data associated with a feeding instance of an infant during a first time period. The computerized data processing system is configured to calculate a baseline pressure from the data, detect sucks by comparing pressure fluctuations to known characteristics, detect swallows by comparing pressure fluctuations to known characteristics, detect respiration by comparing temperature fluctuations to known characteristics, calculate a second time period between the sucks and the swallows to detect bursts, calculate a third time period without the sucks and without the swallows, calculate at least one biomarker for the infant or the feeding instance (e.g., oral cavity pressure changes or respiratory changes by temperature variations), analyze the at least one biomarker to track maturation and neuro-development of the infant (which allows for an identification if early intervention is needed for the infant), and transfer results of the feeding instance to at least one of an engine executable on a computing device, a cloud, a graphical user interface (GUI) of the computing device, or a telehealth interface of the computing device. The results of the feeding instance comprise curves depicting at least one of feeding trace data, maturation progress data, duration of quality data, and recovery data.

In some examples, the computing device comprises a telehealth interface such that a user may interact with the telehealth interface to transmit the results to a third-party. In some embodiments, the computerized data processing system includes one or more algorithms that are configured to: smooth the data and separate the sucks from the swallows, select single and multiple suck signals in a suck/swallow sequence, and a detect a correlation between the suck/swallow sequence and respirations.

Moreover, subsequent establishing the baseline pressure from the data, the computerized data processing system is further configured to determine an amplitude of suck, swallow and respiration curves and detect a difference in a shape of a suck curve as compared to a swallow curve. In some examples, the baseline pressure is calculated from the data by taking a measurement at a beginning and an end of the feeding instance. In other examples, the baseline pressure is calculated by taking a measurement at the beginning and at the end of every burst during the feeding instance.

In some examples, the device also includes a pyroelectric detector or a pyroelectric film that is further configured to collect a respiration measurement. The computerized data processing system is also configured to analyze the respiration measurement, compare the respiration measurement to measurements taken during the feeding instance when the infant is engaging in sucking and swallowing actions, and determine a frequency or an absence during the sucking and swallowing actions, a quality of a shape, and a comparison of the frequency and a strength during a burst and during a rest period.

In some examples, the at least one sensor comprises the respiratory sensor. The respiratory sensor is part of the embedded electronic components. Further, the respiratory sensor is configured to measure changes in temperature in exhalation. In other examples, the at least one sensor includes the pressure sensor. The pressure sensor is located inside of the bottle. The pressure sensor is configured to measure an oral cavity pressure of the infant. In other examples, the at least one sensor includes both the respiratory sensor and the pressure sensor.

In other examples, the device functions independently of an external system. In some examples, the device functions in conjunction with an external system. Moreover, in some examples, the embedded electronic components are further configured to differentiate each of the sucks as a nutritive suck or a non-nutritive suck. In other examples, the embedded electronic components are further configured to assess a ratio of suck to swallow in both count and strength, clarity of signal, and coordination between suck/swallow and respiration.

It is an objective of the present invention to provide a device for measuring infant feeding performance.

It is an objective of the present invention to provide a device that screens infant feeding performance before the infant leaves the hospital to improve immediate care and identify post-discharge needs for the infant.

It is an objective of the present invention to provide a commercially useful clinical tool that captures quantitative feeding parameters of high-risk infants.

It is an objective of the present invention to provide a device that measures the complex changes in oral cavity structure (e.g., jaw, mouth, tongue, palate, esophagus, and/or throat) of an infant through changes in oral cavity pressure, respiration, or recovery.

It is an objective of the present invention to provide a device that enables caregivers to optimize discharge protocols for an infant, monitor the infant remotely, and determine the optimal care for the infant to reduce complications and hospital readmissions due to poor weight gain.

It is an objective of the present invention to provide a device that quantifies oral feeding and supports clinical decision making.

It is an objective of the present invention to provide a device that quantifies the changes in infant development and supports clinical decision making.

### BRIEF DESCRIPTION OF THE DRAWINGS

FIG. 1 depicts a schematic diagram of a device for measuring infant feeding performance, the device being associated with U.S. Pat. Nos. 8,413,502 B2 and 8,473,219 B2, the entire contents of which are hereby incorporated by reference in their entirety.

FIG. 2A depicts a schematic diagram of a shallow angle for a respiratory sensor of a device, according to at least some embodiments disclosed herein.

FIG. 2B depicts a schematic diagram of a steep angle for a respiratory sensor of a device, according to at least some embodiments disclosed herein.

FIG. 2C depicts a schematic diagram of the placement of a respiratory sensor over, or within, the collar of the nipple as part of a device, according to at least some embodiments disclosed herein.

FIG. 2D depicts an image using a specific angle for a respiratory sensor of a device, according to at least some embodiments disclosed herein.

FIG. 2E depicts another image using a specific angle for a respiratory sensor of a device, according to at least some embodiments disclosed herein.

FIG. 2F depicts a further image using a specific angle for a respiratory sensor of a device, according to at least some embodiments disclosed herein.

FIG. 2G depicts another image using a specific angle for a respiratory sensor of a device, according to at least some embodiments disclosed herein.

FIG. 2H depicts an image of a respiratory sensor within a collar of the nipple as part of a device, according to at least some embodiments disclosed herein.

FIG. 3A depicts a block diagram of a system containing the device of FIG. 1 for measuring infant feeding performance, according to at least some embodiments disclosed herein.

FIG. 3B depicts another block diagram of a system containing the device of FIG. 1 for measuring infant feeding performance, according to at least some embodiments disclosed herein.

FIG. 3C depicts a further block diagram of a system containing the device of FIG. 1 for measuring infant feeding performance, according to at least some embodiments disclosed herein.

FIG. 4A depicts a block diagram of a method, according to at least some embodiments disclosed herein.

FIG. 4B depicts another block diagram of a method, according to at least some embodiments disclosed herein.

FIG. 5 depicts a chart depicting results of a peak detection method, according to at least some embodiments disclosed herein.

FIG. 6 depicts two graphs showing detected peaks using a peak detection method, according to at least some embodiments disclosed herein.

FIG. 7 depicts another graph showing detected peaks using a peak detection method, according to at least some embodiments disclosed herein.

FIG. 8 depicts a graph showing an inverted signal and detected troughs, according to at least some embodiments disclosed herein.

FIG. 9 depicts two graphs showing raw data with a mean, according to at least some embodiments disclosed herein.

FIG. 10 depicts a graph with one method of determining base pressure normalized and used for peak detection, according to at least some embodiments disclosed herein.

FIG. 11 depicts a graph utilizing matched filter analysis, according to at least some embodiments disclosed herein.

FIG. 12 depicts another graph demonstrating the different types of peaks that can be captured and the need to be distinguished from a suck, according to at least some embodiments disclosed herein.

FIG. 13 depicts a further graph using a moving average for peak detection, according to at least some embodiments disclosed herein.

FIG. 14 depicts a schematic diagram of data flow of a system, according to at least some embodiments disclosed herein.

FIG. 15 depicts a block diagram of a computing device included within the system of FIG. 3A and/or FIG. 3C, according to at least some embodiments disclosed herein.

FIG. 16 depicts a graph of the behavior associated with a suck-swallow feeding trace of a neonate, according to at least some embodiments disclosed herein.

FIG. 17A depicts an expanded view of the graph of FIG. 16, showing peak type differences, according to at least some embodiments disclosed herein.

FIG. 17B depicts another expanded view of the graph of FIG. 16, showing peak type differences, according to at least some embodiments disclosed herein.

FIG. 17C depicts another expanded view of the graph of FIG. 16, showing peak type differences, according to at least some embodiments disclosed herein.

FIG. 17D depicts a further expanded view of the graph of FIG. 16, showing peak type differences, according to at least some embodiments disclosed herein.

FIG. 17E depicts another expanded view of the graph of FIG. 16, showing peak type differences, according to at least some embodiments disclosed herein.

FIG. 17F depicts a further expanded view of the graph of FIG. 16, showing peak type differences, according to at least some embodiments disclosed herein.

FIG. 18 depicts a graph of the behavior associated with a suck-swallow feeding trace with bursts of another neonate, according to at least some embodiments disclosed herein.

FIG. 19A depicts an expanded view of the graph of FIG. 18, depicting peak and trough variations, according to at least some embodiments disclosed herein.

FIG. 19B depicts another expanded view of the graph of FIG. 18, depicting peak, troughs, and baseline variations, according to at least some embodiments disclosed herein.

FIG. 20 depicts a graph illustrating organized suck, swallow, and breathing per second, according to at least some embodiments disclosed herein.

FIG. 21 depicts a graph illustrating a feeding maturity curve, according to at least some embodiments disclosed herein.

FIG. 22 depicts a graph illustrating quality assessment curve/analysis, according to at least some embodiments disclosed herein.

FIG. 23 depicts a first graph illustrating disorganized/erratic feeding patterns, a second graph illustrating organized feeding patterns, and a third graph illustrating evaluation over time or feeding consistency, according to at least some embodiments disclosed herein.

FIG. 24 depicts three graphs illustrating examples of neonatal abstinence syndrome (NAS) recovery, according to at least some embodiments disclosed herein.

FIG. 25 depicts an additional three graphs illustrating examples of NAS recovery, according to at least some embodiments disclosed herein.

FIG. 26 depicts an additional two graphs illustrating examples of NAS recovery, according to at least some embodiments disclosed herein.

FIG. 27 depicts a graph illustrating respiration and oral cavity changes associated with immature feeding, according to at least some embodiments disclosed herein.

FIG. 28 depicts a graph illustrating respiration and oral cavity changes associated with developing feeding, according to at least some embodiments disclosed herein.

FIG. 29 depicts a graph illustrating respiration and oral cavity changes associated with mature feeding, according to at least some embodiments disclosed herein.

FIG. 30 depicts a graph illustrating several measured oral cavity biomarkers that are used together to assess a developmental status for an infant, according to at least some embodiments disclosed herein.

FIG. 31 depicts graphs illustrating using patterns in oral feeding and the tracking of maturation/neuro-development, according to at least some embodiments disclosed herein.

FIG. 32 depicts graphs showing variables on a bench that need to be managed, according to at least some embodiments disclosed herein.

### DESCRIPTION OF THE PREFERRED EMBODIMENTS

The preferred embodiments of the present invention will now be described with reference to the drawings. Identical elements in the various figures are identified with the same reference numerals. Reference will now be made in detail to each embodiment of the present invention. Such embodiments are provided by way of explanation of the present invention, which is not intended to be limited thereto. In fact, those of ordinary skill in the art may appreciate upon reading the present specification and viewing the present drawings that various modifications and variations can be made thereto.

While there are many issues surrounding the survival and health of newborns, the need for adequate nutrition is fundamental. Over 50 percent of the 13 million preterm infants born globally each year (approximately 600,000 in the U.S.), as well as a significant portion of the 640,000 U.S. infants born to mothers with addiction, 270,000 infants born to mothers with diabetes, and 43,000 infants born with congenital anomalies will have associated feeding problems that threaten their survival and growth. Clinical decision making for initiating and advancing oral feeding for infants remains a major challenge due to the reliance on descriptive and subjective information regarding an infants' sucking skills.

One group has reported more than 50% of NICUs do not have a specific policy for initiating oral feeding. (Siddell, 1994). Feeding infants when they are not ready has been shown to increase stress on the infant, slow progression of feeding, and delay the discharge of the infant. (Carty, 2018). For all high-risk infant populations, feeding problems results in increased healthcare utilization. (Capilouto 2016). Infants at the greatest risk for feeding difficulties are high-risk infants in the Level IV NICU, which include extremely (<30 weeks) preterm infants and infants who experience neonatal surgery for complex congenital heart disease. (Mussatto, 2014).

Infants develop unique nursing skills and anatomy just prior to a full-term birth. In general, feeding in an infant requires brainstem/subcortex driven oral cavity skills to ensure efficient nutrient consumption and safe respiratory protection, and, as such, is a recognized critical and developmental skill. Thus, the basic ability to breast or bottle-feed is not necessarily a fully developed, inborn skill not necessarily developed in the preterm or developmentally delayed infant. Newborns who have feeding problems are at much greater risk for malnutrition and the resulting growth failure. Ample evidence shows growth failure leads to a range of behavioral, cognitive, language, and motor development disabilities and an increased risk for infectious disease. In fact, infants who have early poor feeding skills are more likely to have lower developmental scores at twelve months of age than infants with appropriate feeding skills. (Simpson, 2002).

However, it is not only premature infants who are considered to be at risk for feeding dysfunction. Congenital heart defects (CHD) are the most common birth defect in the United States, affecting 40,000 births each year and often requiring surgery. (Medoff-Cooper, 2011). It has been observed that almost 50% of all infants who underwent neonatal cardiac surgery experienced feeding dysfunction as a result of anesthesia, and possible insults to their pharynx, trachea, vocal cords, and phrenic nerve due to mechanical ventilation and surgery (Medoff-Cooper, 2020). Typically, infants who experience feeding dysfunction during hospitalization are often discharged with a feeding tube in place. Managing a feeding tube is stressful for parents, since there are currently no objective markers to indicate when the infant has sufficiently recovered to successfully orally feed. While observational methods developed for monitoring feeding competence have helped to define the nature of the problem, they are only implemented properly when use by highly-trained specialists in research hospitals.

The main goal of feeding is the acquisition of sufficient nutrients for optimal growth and development. Malnutrition may result directly from feeding problems and may also perpetuate them. (Stevenson, 1991). Successful newborn feeding is dependent on infant sucking skills. Given as many as 25%-45% of normally developing children and 40-70% of premature infants exhibit both immature and atypical feeding patterns. As such, it is imperative that clinicians have a device to measure feeding patterns for those infants who are not demonstrating adequate feeding skills in the newborn period. (Cerro, 2002). (Hawdon, 2000).

A healthy full-term infant has developmentally appropriate anatomy and reflexes to coordinate the complex process of feeding. This process requires the coordination of sucking, swallowing, and breathing. Nutritive sucking is initiated in utero and continues to develop in an organized pattern in the early weeks after birth. It involves the integration of multiple sensory and motor central nervous system functions. (Wolff, 1966).

Sucking activity uses the whole mouth and jaw in a process to produce a negative intra-oral pressure, together with expression/compression of the nipple between the baby's tongue and the hard palate in a rhythmic fashion. (Lau, 2000). Further, as infants develop they are known to have variability in their ability to coordinate sucks to swallow. In the process, the number of sucks per swallow to create a successful transfer of a bolus of nutrient changes. (Gewolb, 2001). Milk boluses then flow from the nipple into the oral cavity towards the throat as the esophagus is opened, from which they are swallowed, while the airway is protected. Sucking and swallowing must be effectively synchronized.

Sucking appears as early as 28 weeks postmenstrual age (PMA), but at this initial stage, there is no coordination between the activities of sucking and swallowing. It is only between 32-34 weeks PMA that nutritive sucking begins to be effective. Sucking episodes are organized into bursts and pauses between bursts. As PMA advances, the frequency and maximal negative pressure of the sucks increase, as well as the length of the sucking bursts. (Medoff-Cooper, 2001). (Lau, 2000). It is suggested that the pattern of nutritive sucking within a feeding provides an important window into the integrity of the developing central nervous system. (Wolff, 1966). Vulnerable infant populations are particularly at risk for disturbed feeding behaviors. (Medoff-Cooper, 2009). (Gewolb, 2004).

Premature birth has been associated with undernutrition and growth faltering. (Rogers, 2003). (Huysman, 2003). An important mediator of the association between preterm infant birth and growth may include feeding skills. The work of Medoff-Cooper and colleagues demonstrated how changes in the pattern of nutritive sucking behaviors can be correlate with gestational age in healthy preterm and full-term infants. (Medoff-Cooper, 2002). (Medoff-Cooper, 2000). This group reported how sucking patterns change systematically with increasing gestational age, with a strong correlation between increasing maturation and more organized sucking patterns. (Medoff-Cooper, 2002).

When comparing sucking behaviors at term of 213 infants having a gestational age of ≤29 weeks, more mature preterm infants (e.g., at 30-32 weeks gestational age), and full-term infants, sucking behaviors were noted to be a function of gestational age at birth, as well as the interaction of maturation and experience. Extremely early born preterm infants demonstrated less competent feeding behaviors than either more mature preterm infants or newly born full-term infants. The measurements/data gleaned by the instant invention provide objective data to track these at-risk infants with three different assessment screens: feeding trace, maturation progress, and duration of quality.

Moreover, infants exposed to opiates during pregnancy experience neonatal abstinence symptoms (NAS), which interfere with infant feeding in a variety of ways. Opiate exposed infants are at risk for feeding problems due to altered sucking patterns. One group compared suck and swallow patterns between drug-exposed and non-drug-exposed infants at 3 days and 1 month of age. (Gewolb, 2004). This group found that intrauterine drug exposure adversely impacted development of the infants brainstem respiratory and swallow centers, which led to a dysregulation of the underlying biorhythms of feeding. At 3 days of age, this group found that drug-exposed infants had more apneic swallows, less breath-breath rhythmic stability, and shorter swallow-breath intervals. They were less efficient feeders and ingested less volume per group of swallows than controls. (Gewolb, 2004).

Furthermore, approximately 50% of infants with complex congenital heart disease will experience adverse nutritional sequelae early in their lives, most often resulting in failure to thrive (FTT). (Forchielli, 1994). FTT is a term used to describe inadequate gains in weight, length or other growth parameters. Feeding dysfunctions are particularly common in children with complex congenital heart disease. (Hehir, 2011). (Jadcherla, 2009). Early sucking and swallowing problems have been shown to be a significant predictor of neurodevelopmental outcomes in children. (Adams-Chapman, 2013). (Slattery, 2012). Neonates with congenital cardiac disease face significant barriers to successfully achieving oral feeding on hospital discharge. Enteral feeding guidelines focus on physiological stabilization and do not always address the developmental milestones necessary to support oral feeding. Once at home, the management of feeding is a source of stress for parents. (Hartman, 2012). Attempts have been made to improve feeding evaluations. However, some methods are subjective and fall short of measuring oral cavity changes needed for sucking and swallowing.

Suck, swallow, and breathing patterns are well known in the literature to change as a neonate matures. The oral cavity experiences many changes during a suck, a swallow, and a breath. The entire shape and volume changes as the tongue of the infant depresses the nipple on the hard palate, the cheeks and lips compress, and the esophagus and the trachea open and close. When capturing oral cavity pressure using a sensor to capture data over time, challenges present themselves. For example, nuances of each anatomical change impact the shape or the curve. Additionally, measurements that capture bubbles in the fluid flow make this even more difficult to differentiate. Further, as an infant latches on, and then proceeds to suck and swallow in continuous action, the pressure within the oral cavity may increase or decrease as residual pressure is maintained within the oral cavity. To be able to measure the change in pressure due to a suck or swallow this must be dynamically adjusted.

The present invention incorporates components/features to capture appropriate pressure variations, and address pressure caused noise in the signal during a reading. Further, the present invention provides a design that incorporates required features in conduit transitions that do not capture or create air bubbles. Further, because residual pressure in the oral cavity is not static, a method to adjust base line pressure dynamically such that the amplitude of the suck and swallow deviations can be measured. The present invention does this by comparing the pressure at the beginning and end of a burst and setting a straight line between the two to measure pressure off of. Further, the present invention dynamically utilizes all of the pressure measurements to compare from one to the other.

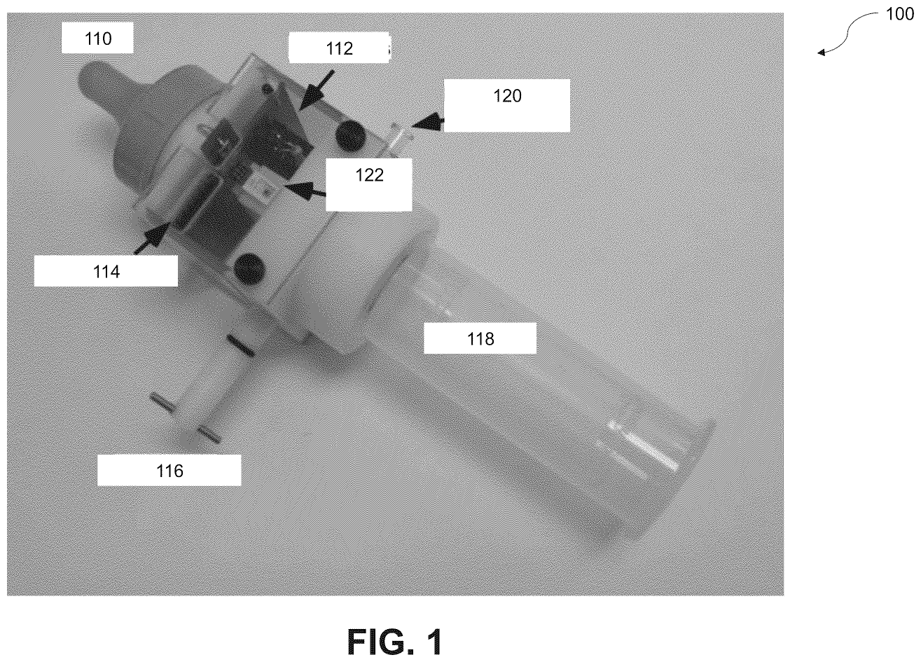

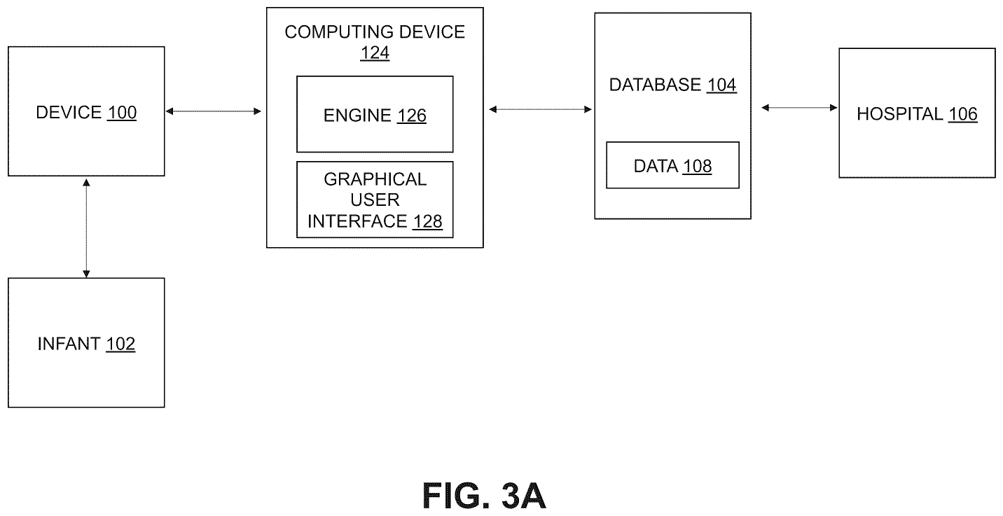

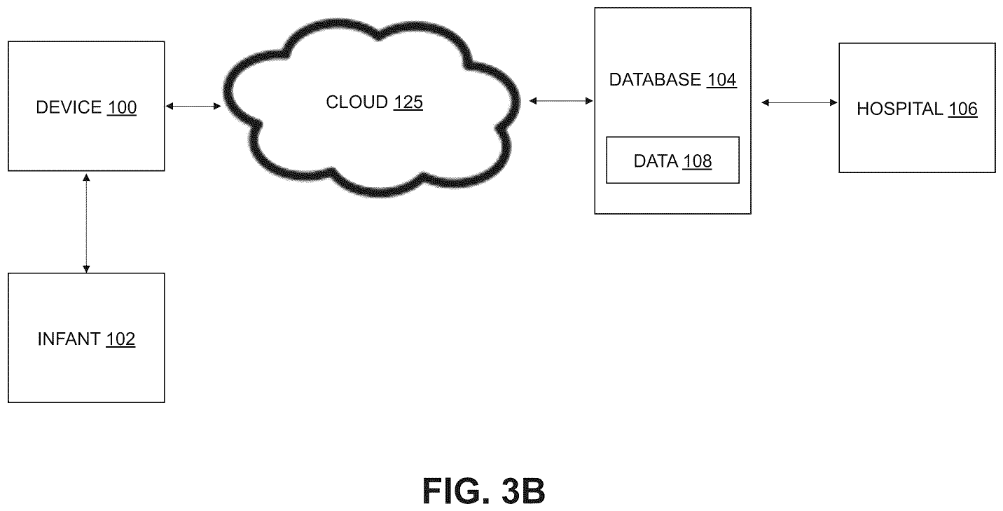

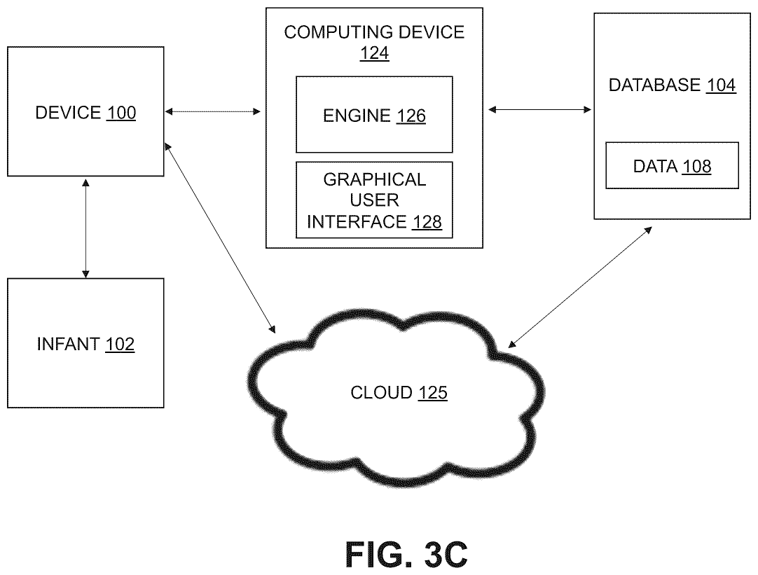

The device 100 of FIG. 1 and FIG. 3A, FIG. 3B, and FIG. 3C described herein is patented in U.S. Pat. Nos. 8,413,502 B2 and 8,473,219 B2, the entire contents of which are hereby incorporated by reference in their entirety. The device 100 is a hand-held device that fits between the nipple and nutrient bottle to measure pressure changes created between a restriction in the flow path between the nutrient filled bottle, the oral cavity mimicking the natural flow resistance from a nipple, and respiration, as air travels past a sensor.

It should be appreciated that the device 100 measures, records, and monitors feeding factors related to feeding performance of an infant 102 of FIG. 3A, FIG. 3B, and FIG. 3C during a feeding session. To measure the feeding factors, the device 100 includes embedded electronics (e.g., electronic components 112). The electronic components 112 include at least one sensor and a computerized data processing system. In one embodiment, the device 100 includes an embedded microcontroller and at least one embedded sensor, such as a pressure sensor 122 and a respiratory sensor, which is part of the electronic components 112, for measuring and recording a feeding factor, such as sucking pressure within and fluid volumetric flow through the device 100. A PC connection 114 is also depicted. The location of the sensors proximate the infants nose is paramount to obtaining accurate readings. It should be appreciated that at least one of the sensors has a specific responsiveness of faster than 1/second. For example, when used in conjunction with a feeding nipple 110 and a fluid reservoir holding a comestible fluid (e.g., a baby bottle 118 containing infant formula, expressed breast milk, pediatric electrolyte solution, or the like), the device 100 may measure and record the sucking response exhibited by the infant 102. The device 100 may function independently of an external system for data collection, recording, and/or analysis. Specifically, the pressure sensor 122 is located inside of the baby bottle 118 and is configured to measure oral cavity pressure. The pressure sensor 122 is an integral part of the flow channel. The respiratory sensor is configured to measure changes in temperature in exhalation—there for the proximity to the nose is critical, the sensitivity and responsiveness (e.g., how fast it can measure change) are all critical.

As explained herein, a “feeding session” refers to a continuous period of time during which the infant 102 (e.g., between birth to about one-year old) is provided with a feeding device, such as the baby bottle 118, and is encouraged to feed from the device 100. The feeding session may be a function of a predetermined period of time, a predetermined consumption volume, or both. A feeding performance of the infant 102 may be determined during the feeding session. The feeding session may also be subdivided into two or more epochs to further quantify the feeding performance of the infant 102. Feeding sessions also can be subdivided in relation to one or more events or markers, such as an instance of particular behavior demonstrated by the observed infant 102.

As explained herein, a “feeding performance” refers to the infants 102 innate or acquired capacity for orally feeding via a synthetic nipple (e.g., the nipple 110), expressed as physical, physiological and/or behavioral responses during the given feeding session.

As explained herein, “feeding factors” relate to one or more physical, physiological, and/or behavioral responses produced or exhibited by the infant 102 while orally feeding or attempting to orally feed. Examples of feeding factors include sucking pressure, expression pressure, oxygen saturation level, swallowing, and respiration, among others not explicitly listed herein.

It should be appreciated that the embedded electronics (e.g., electronic components 112) may differentiate between a nutritive suck and a non-nutritive suck.

As used herein, “nutritive suck” refers to the process of a subject (“subject”) feeding with a bottle or breast and receiving fluid. Therefore, a “nutritive suck” (“NS”) condition is one where the nipple element and/or the apparatus including the instrumented nipple is configured such that liquid is passed through the nipple during a sucking event. For example, the NS nipple may define an aperture through which liquid in communication with the nipple aperture, which may be liquid in a bottle or other container to which the NS nipple is attached, may flow through the nipple into a subject's oral cavity during a sucking or feeding event. In nutritive suck the fluid is typically a substance ingested by the infant during feeding, such as infant formula, water, milk, etc. As used herein, fluid type is not meant to be limiting. An element of the NS is the swallowing that also is detected.

As used herein, “non-nutritive suck” refers to the process of a subject (“subject”) performing the same task as nutritive feeding but not receiving fluid. A “non-nutritive suck” (“NNS”) condition is one where any liquid does not need to be delivered, and the if it is, the liquid is not swallowed e.g., no feeding occurs. The nipple in NNS may contain an aperture for passage of fluid or may be sealed. In a non-limiting example, a NNS nipple may be configured without an aperture such that fluid flow through the nipple is prevented. In another example, a NNS nipple may be configured as a pacifier. In another example, an evaluation apparatus may include a nipple with an aperture which may be used in either of a NS (liquid provided) condition or NNS (no liquid provided) condition.

As shown in FIG. 1, between the nipple 110 and the baby bottle 118 is the device 100, containing a duckbill valve air inlet 120, the electronic components 112, the PC connection 114, the one or more sensors 122, and a valve stem 116. The pressure sensor of the one or more sensors 122 is glued into the holder and the combined unit is inserted into an access hole. The electrical contact to the pressure sensor of the one or more sensors 122 occurs when the electronic components 112 are inserted into the system.

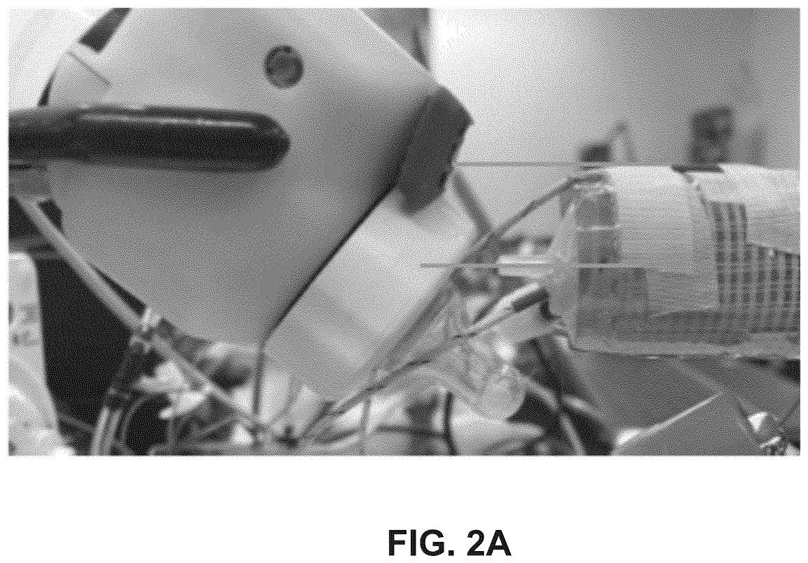

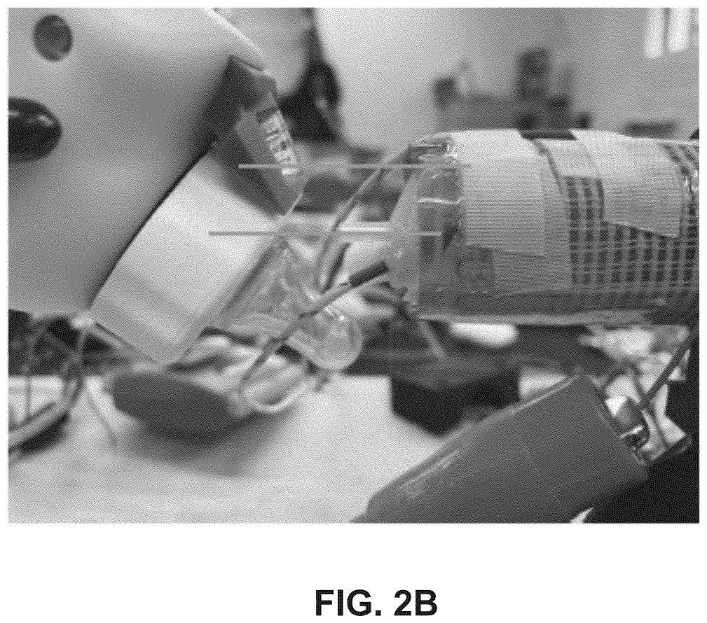

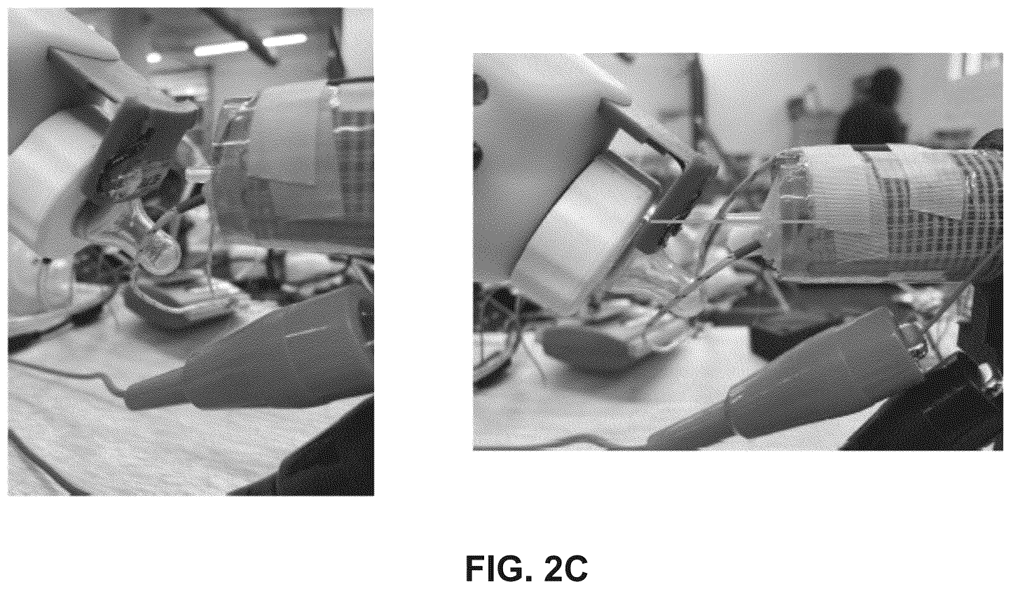

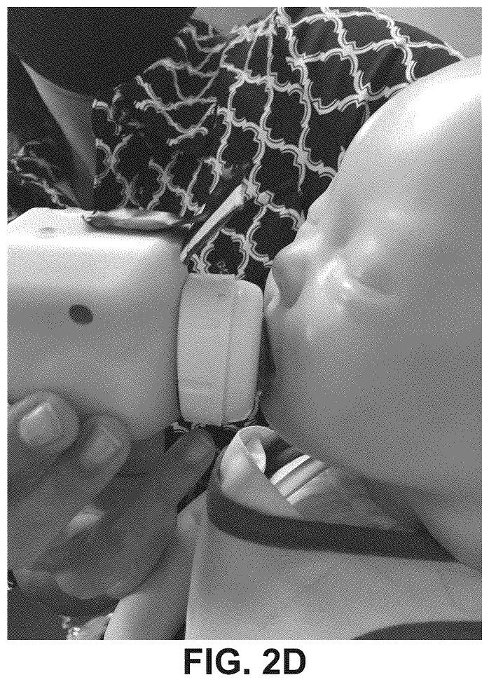

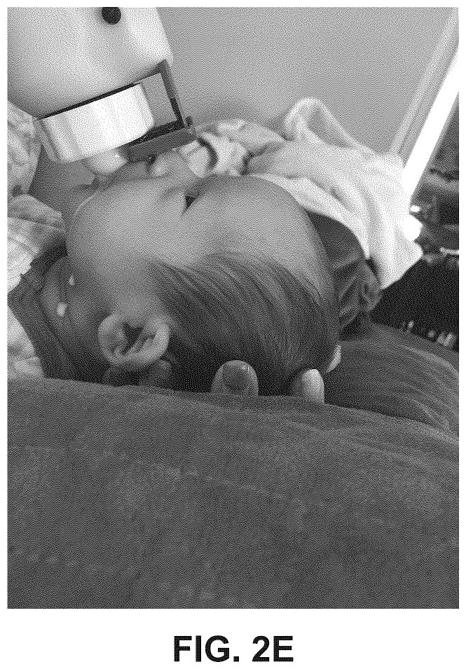

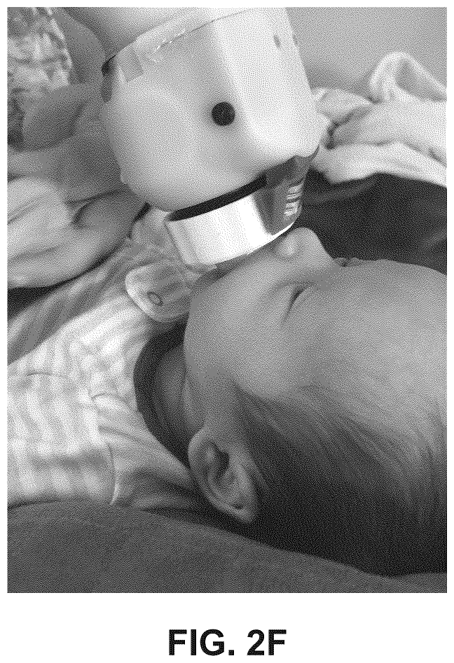

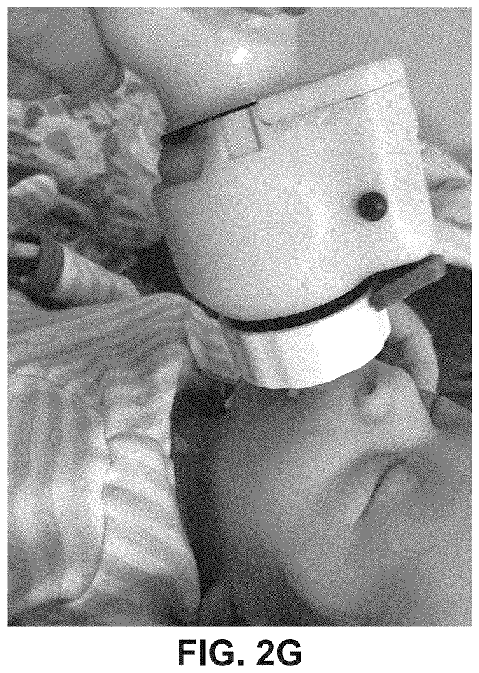

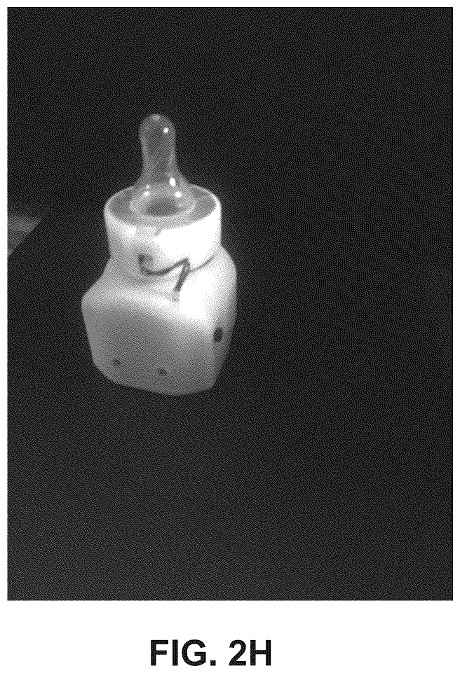

FIG. 2A depicts a schematic diagram of a shallow angle for the respiratory sensor of the one or more sensors 122, FIG. 2B depicts a schematic diagram of a steep angle for the respiratory sensor of the one or more sensors 122, and FIG. 2C depicts a schematic diagram of a respiratory sensor or the one or more sensors 122 in a design that places it over the collar that holds the nipple. It should be appreciated that the location for the respiratory sensor must be adjusted as the size of an infant from early prematurity (e.g., 23 weeks gestational age—or an age after the egg is fertilized) is different from a full term, or older infant. The location of the respiratory sensor of the one or more sensors 122 in FIG. 2A, FIG. 2B, and FIG. 2D is associated with a 34 week old infant. The placement of the sensor such that it is on top of or embedded into the collar of FIG. 2C and FIG. 2E is used when the location of the premature infant is smaller. FIG. 2A, FIG. 2B and FIG. 2D are used for larger/older infants. The pressure sensor of the one or more sensors 122 in FIG. 2C is located near a top plane of a collar and is in line with a smaller, e.g., premature infant nose/mouth position.

It should be appreciated that in some embodiments, the respiratory sensor of the one or more sensors 122 may be a pyroelectric detector, which is particularly sensitive to temperature changes, and can provide measurements with a degree of speed faster than 20/second, without heating. Moreover, in some implementations, the pressure sensor of the one or more sensors 122 may be part of the collar component (e.g., FIG. 2H), and used with or without measuring feeding.

FIG. 2D-FIG. 2G depict images using differing angles for the respiratory sensor (located between the electronic components 112 and the PC connection 114) of the device of FIG. 1. These images show the challenges associated with getting the device of FIG. 1 under a nose of a premature infant. As the infant 102 grows, the respiratory sensor may need to move to a location in which a larger infant 102, or child, breaths on the respiratory sensor, and hence why the different locations and styles are important and contemplated by Applicant herein.

FIG. 3A, FIG. 3B, and FIG. 3C depict block diagrams of systems containing device 100 for measuring infant 102 feeding performance. Specifically, when the infant 102 interacts with the device 100, the pressure sensor of the one or more sensors 122 of the device 100 is configured to gather and transmit data 108 to a receiver (which may be housed in a computing device 124). The computing device 124 may be a computer, a laptop computer, a smartphone, and/or a tablet, among other examples not explicitly listed herein.

In examples, the computing device 124 may include an engine 126 that is configured to interact with a database 104. The database 104 houses data 108. A third-party, such as a hospital 106, may interact with the database 104 or a presentation and graphical interface to view and/or manipulate the data 108. In some examples, the data 108 may be sent to an electronic medical record (EMR) or a simulated EMR for clinician evaluation.

The engine 126 may receive, analyze, and/or manipulate data from the device 100. In some examples, the engine 126 may be an application, a software program, a service, or a software platform configured to be executable on the computing device 124. The engine 126 includes a customized means to interpret data to separate out suck from swallow signals, a customized means to establish a baseline for assessments, a customized means to select single and multiple suck signals (in a suck/swallow sequence), and a customized means to detect correlation between suck/swallow patterns and respirations. Establishing a baseline for assessments is essential to: (1) determine an amplitude of both suck, swallow and respiration curves and (2) detect a difference in shape of a suck curve as compared to a swallow curve. In some implementations, the engine 126 may utilize one or more artificial intelligence and/or machine learning algorithms to perform one or more actions described herein.

There are multiple ways that the baseline pressure can be measured. For example, in a first method, the baseline pressure is measured by taking a measurement at a beginning and an end of the sample, before any suck occurs by the infant 102. In another example, the baseline pressure is measured by taking a measurement at the beginning and the end of every burst. It should be appreciated that the display can either be an actual measurement or an adjusted measurement to account for baseline pressure corrections.

According to the invention described herein, respiration is detected by a change of temperature as the infant 102 exhales, and inhales out of their nose. The “respiration” measurement may be collected using the pyroelectric detector or a pyroelectric film, or other fast acting sensor (>20 points per second) that does not generate heat. Then, the respiration measurement is analyzed by the engine 126, which then compares the respiration measurement to measurements taken when the infant 102 is engaging in sucking and swallowing actions. As such, the engine 126 is configured to determine a frequency or absence during the suck and swallow actions, a quality of the shape, and a comparison of the frequency and strength during the burst and during a rest period.

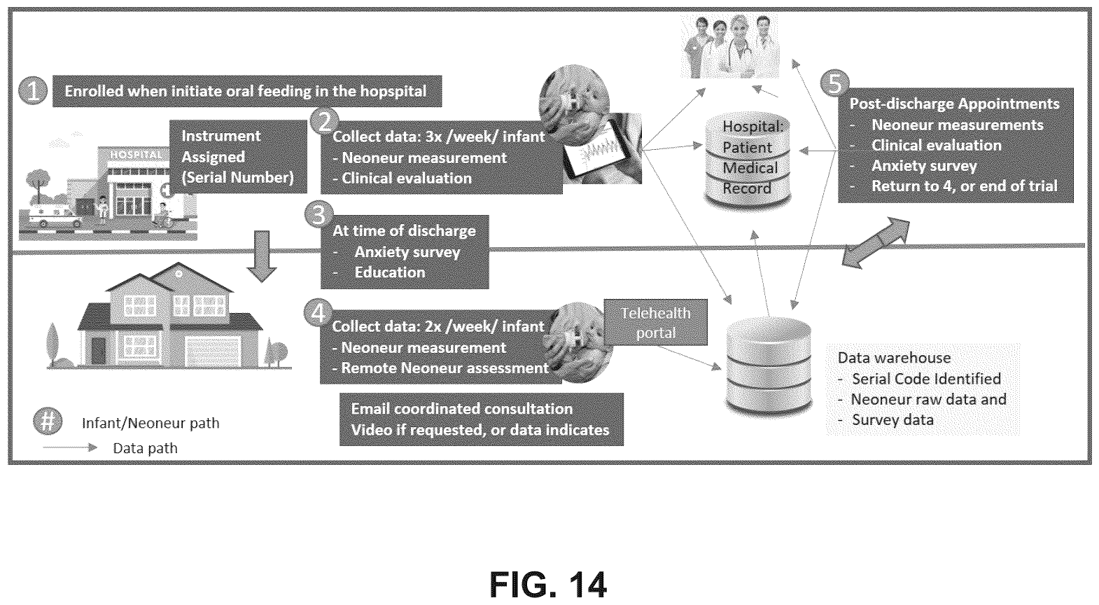

The computing device 124 may also include a graphical user interface (GUI) 128. A user may interact with the engine 126 via the GUI 128 to view or edit the data 108. Further, the engine 126 may be configured to receive login credentials from the user, such as a username, a password, a biometric identification means (e.g., fingerprint identification, face recognition identification, palm print identification, iris recognition, retina recognition, etc.), etc. and information about the subject for who feeding data is collected. In response, the engine 126 may compare the login credentials of the user and subject to information stored in the database 104 to determine an identity of the user. Identification of the user may include information such as: a name of the user, a telephone number of the user, an address of the user, an access level granted to the user, etc. The access level may be a first access level or a second access level, where the first access level provides the user with read-only credentials associated with the data 108 and the second access level allows the user to interact with or edit the data 108, as well as interact with a telehealth interface. In some instances, the user may interact with the telehealth interface executed on the computing device 124 to transmit the data 108 to the third-party (e.g., the hospital 106). Telehealth care is personalized healthcare delivered over a distance with data transferred from the patient to the professional. Specifically, examples of this data flow is shown in FIG. 14. HIPPA control is managed by only sending patient de-identified data, such as serial number, date, and raw text data.

Though the computing device 124 is described and depicted in FIG. 3A and FIG. 3C, in some embodiments, the database 104 may comprise the receiver such that the sensors 122 of the device 100 may directly transmit the data 108 to the database 104. In some examples, the process steps may occur in a cloud 125 (e.g., FIG. 3B). In other examples, the process steps may occur in the computing device 124 and in the cloud 125 (e.g., FIG. 3C). Further, in some examples, the hospital interface is not needed.

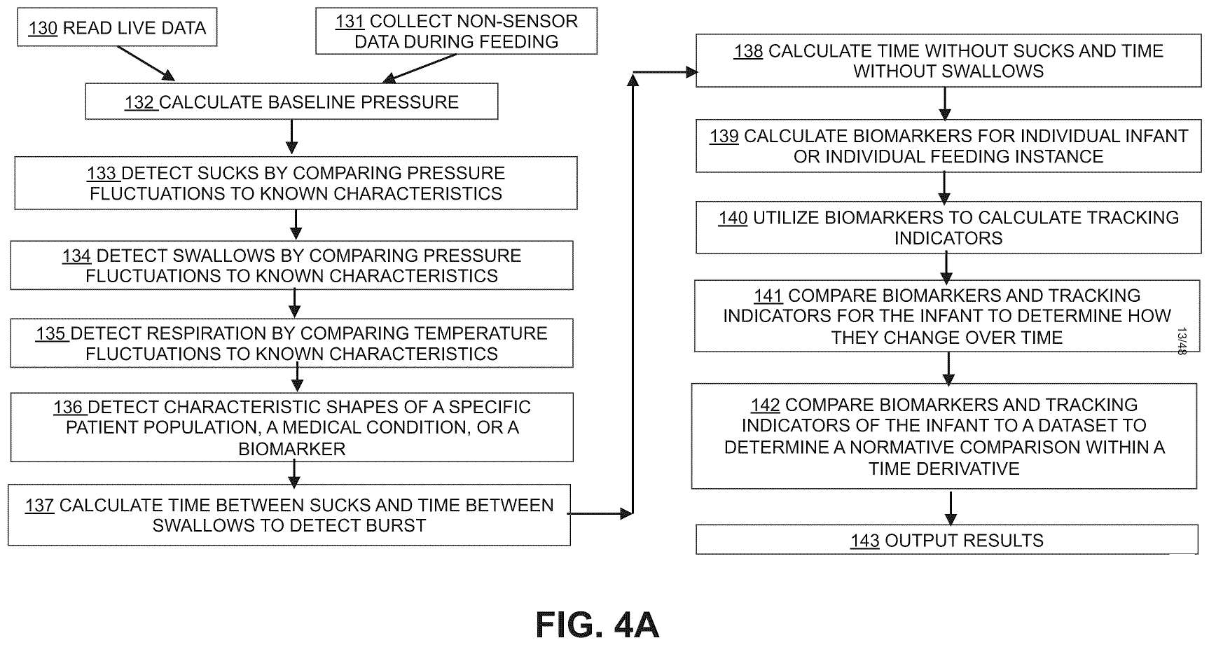

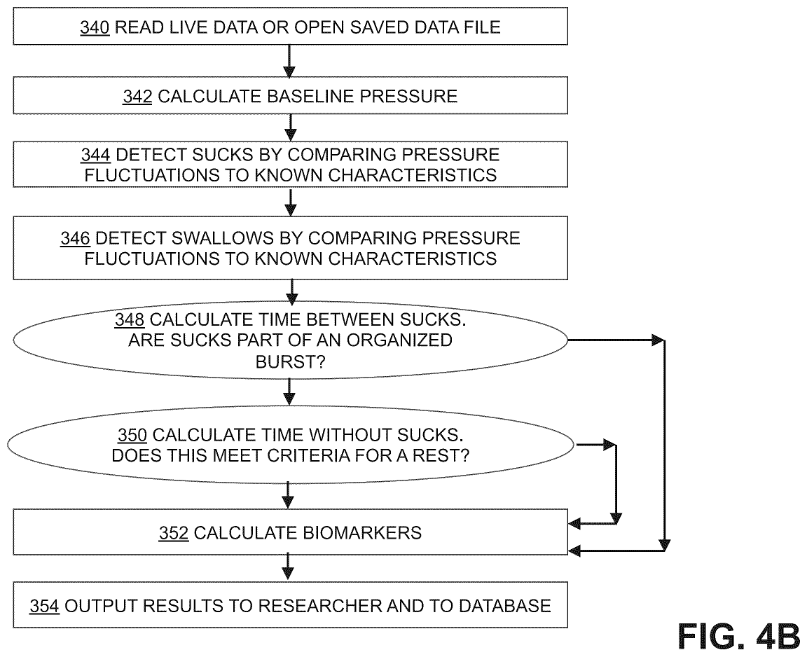

FIG. 4A depicts a block diagram of a method executed by the engine 126 of FIG. 3A and FIG. 3C. The method of FIG. 4A includes numerous process steps. For example, the method of FIG. 4A begins at a process step 130, where the engine 126 is configured to read live data from the device 100. A process step 131 also occurs, where the engine 126 is configured to collect non-sensor data during feeding. For example, at this process step, the engine 126 may be configured to receive, via the GUI 128 of the computing device 124, an identifier associated with the infant 102 from a user. A process step 132 follows, where the engine 126 is configured to calculate a baseline pressure from the live data.

A process step 133 follows the process step 132 and includes the engine 126 detecting a quantity of sucks by comparing pressure fluctuations to known characteristics. In some implementations, this process step may include adapting the baseline pressure to an actual reading.

A process step 134 follows the process step 132 and includes the engine 126 detecting a quantity of swallows by comparing the pressure fluctuations to known characteristics. . . . In some implementations, this process step may include adapting the baseline pressure to an actual reading.

A process step 135 follows the process step 132 and includes the engine 126 detecting respiration by comparing temperature fluctuations to known characteristics.

A process step 136 follows the process step 133, 134, or 135 and includes the engine 126 detecting characteristic shapes of a specific patient population, a medical condition, or a given biomarker. Next, a process step 137 follows the process step 136 and includes the engine 126 calculating a time period between the sucks and a time period between the swallows to detect bursts. Then, a process step 138 follows the process step 137 and includes the engine 126 calculating a time period without sucks and a time period without swallows. This process step helps assess if the time period meets the criteria for a rest.

Next, a process step 139 follows the process step 138 and includes the engine 126 calculating biomarkers for the individual infant or for an individual feeding instance. A non-exhaustive list of the biomarkers includes: a maximum pressure, a suck frequency within a burst, a quantity of bursts per time frame (e.g., a first period, such as every two minutes), an inter-suck width (e.g., a size of a suck width as compared to a size of a swallow width at a baseline pressure), a quantity of sucks, sucks/bursts, IBW, and Pmax, among others.

Moreover, the primary biomarker data collected includes (1) oral cavity pressure changes (suck and swallow) and (2) respiratory changes by temperature variations. During a feeding episode, time domain data extracted from primary data includes: data per time period, a consistency component within a set time period (e.g., standard deviation), a variation from a start to an end of a set time period measured intermittently throughout or at the start and the end of the feeding episode, and where it can be a suck, swallow or breath. Additional biomarkers that may be measured during the feeding episode include a burst component/second (e.g., a group of sucks in a rhythmic pattern, before a clear break)/set time period such as 2 minutes), suck components/burst/set time period, swallows/bursts/set time period minutes, a component/second within a burst, an average rest time period between bursts/set time period, breaths/seconds in a rest time period, and an average component shape (e.g., apex with a point or smooth, with a tail, width at baseline, etc.)/time. During a feeding episode, numerous components may be measured, such as: sucks/swallows, breaths/suck, suck strength/swallow strength (e.g., pressure), and a combination of actions (e.g., three positive pressure peaks and one tailed pressure peak correlates to a tongue, tongue, tongue, cheek movement) per burst, or per feeding episode. Assessment of infant 102 patterns in oral feeding allows for the tracking of maturation/neuro-development of the infant 102. This allows one to identify which infants need early intervention.

Moreover, as described herein, “maturation” is a change in a single or a combination of biomarkers used in a statistical grouping, or artificial intelligence (AI), to aligned with the change in infant age. Maturation can be differently affiliated with gestational age (e.g., age of in utero development at birth), or conceptual age (cumulative gestational age and actual age after birth) for infants born premature or full term with no other known abnormality. Further, maturation can be differently affiliated with infant development due to infant developmental capability of infant due to weight at birth, genetic complication, birth abnormality.

Further, as described herein, “recovery” is a change in a single or combination of biomarkers used in a statistical grouping, or AI, aligned with infant recovery to a more healthy status from an injury, illness, or malady.

Next, a process step 140 follows the process step 139 and includes the engine 126 utilizing the biomarkers to calculate tracking indicators. Then, a process step 141 follows the process step 140 and includes the engine 126 comparing the biomarker sand the tracking indicators for the given infant to determine how they change over time. Next, a process step 142 follows the process step 141 and includes the engine 126 comparing the biomarkers and the tracking indicators of the infant to a dataset to determine a normative comparison within a time derivative.

Further, a process step 143 follows the process step 142 and includes the engine 126 outputting results of the method of FIG. 4A. The results may include text, graphs, or charts. In examples, the results may be displayed via the GUI 128 of the computing device 124 or to an external display. The method of FIG. 4A may also include the engine 126 transmitting the results to the database 104.

Further, it should be appreciated that some of these steps may be omitted in specific instances or the order of the process steps may be modified. For example, in one embodiment, the process step 134 may occur alone. In another embodiment, the process step 135 may occur alone. In another instance, the process step 133 and the process step 134 may occur together. In a further example, the process step 133 and the process step 135 may occur together. In another example, the process step 134 and the process step 135 may occur together. It should be appreciated that these examples are provided for illustrative purposes only and the process steps are not limited to such.

In some examples, the results may include a quantitative developmental status of the infant 102 or a quantitative feeding status (e.g., immature feeding, developing feeding, or mature feeding). In preferred examples, the results may include curves depicting feeding trace data, maturation progress data, and/or duration of quality data. The feeding trace information may include: an average number of sucks/bursts, an average length of time between bursts, a frequency of sucks in first 2 minutes, a normal respiratory rate, and an overall duration of feed. The maturation progress information may allow for curve tracking, slope tracking, and speed of change. The duration of quality information may include a score for quality in a first time period (e.g., about two minutes) of a feed and a length of time the quality is maintained.

In some embodiments, the engine 126 may receive data from a third-party, such as: components of oral feeding evaluation (e.g., from a feeding therapist), clinician evaluation of feeding quality (e.g., from the feeding therapist), an anthropometric evaluation (e.g., weight, height, and head circumference data) completed prior to discharge and post-discharge at a follow-up visit or from their pediatrician's office, etc. . . . In some examples, the components of oral feeding evaluation may include: (1) variables (e.g., volume of nutrient by modality (e.g., oral, ng, and GI), frequency of feeding, length of time spent on a feed, nutritional content, nipple size and style, and viscosity), (2) performance (e.g., duration of feeds, strength of suck, sucks/minute, and sucks/burst), (3) adherence/direction (e.g., each variable as part of the ordered plan/actual measure/recommended changes), and (4) an audio or other collected timestamp of duration of quality, pauses, and total length of feeding. The clinical evaluation of feeding quality may include a graded score associated with a quality in a time period (e.g., two minutes) and a graded (1-10) score associated with safety, readiness to attempt full oral feeding, and feeding readiness for discharge. It should be appreciated that other numbering or scores may be used in the clinical evaluation of feeding quality.

In some examples, the process step 133, the process step 134, and/or the process step 135 are optional process steps. Moreover, in some examples, the process step 137 and/or the process step 138 are not required and are merely examples.

FIG. 4B depicts another block diagram of a method executed by the engine 126 of FIG. 3A and FIG. 3C. The method of FIG. 4B begins at a process step 340, where the engine 126 reads live data or opens a saved data file. Next, a process step 342 occurs where the engine 126 calculates a baseline pressure. Then, a process step 344 occurs where the engine 126 detects sucks by comparing pressure fluctuations to known characteristics. Next, a process step 346 occurs where the engine 126 detects swallows by comparing pressure fluctuations to known characteristics. The engine 126 then, in a process step 348, calculates a time between sucks to determine if the sucks are part of an organized burst. Subsequent this step, a process step 352 occurs where the engine 126 calculates biomarkers. Moreover, the engine 126 executes a process step 350 where the engine 126 calculates a time without sucks to determine if this meets criteria for a rest. Subsequent the process step 350, the engine 126 executes the process step 352. Further, a process step 354 occurs where the engine 126 outputs results to researchers and to a database. The process step 354 concludes the method of FIG. 4B.

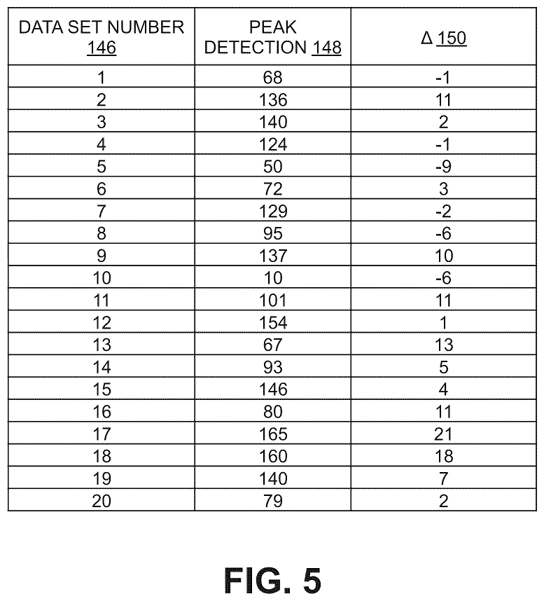

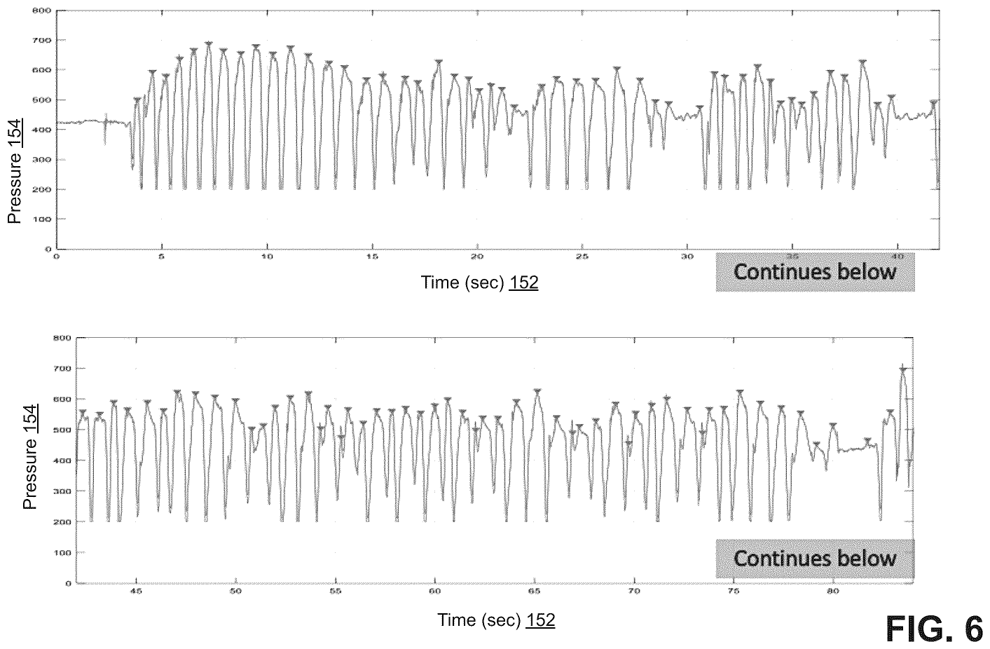

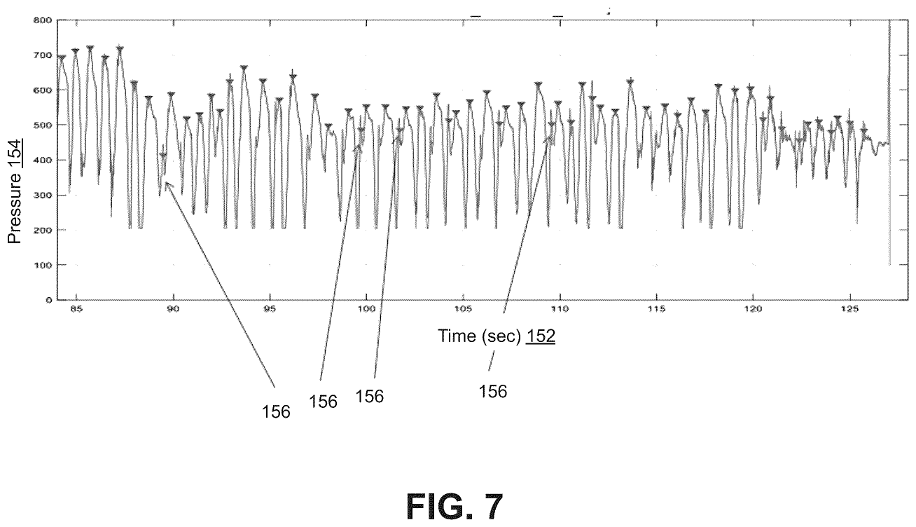

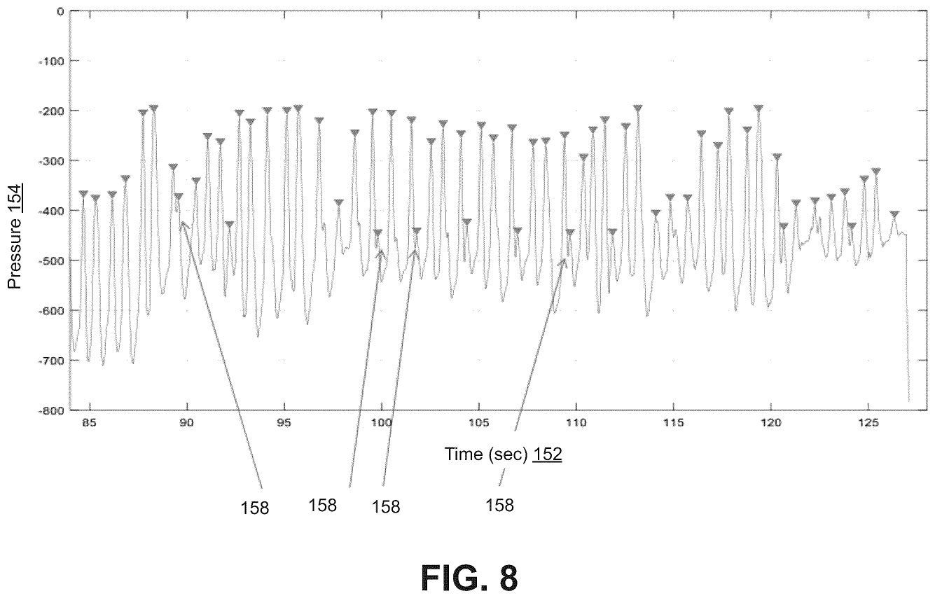

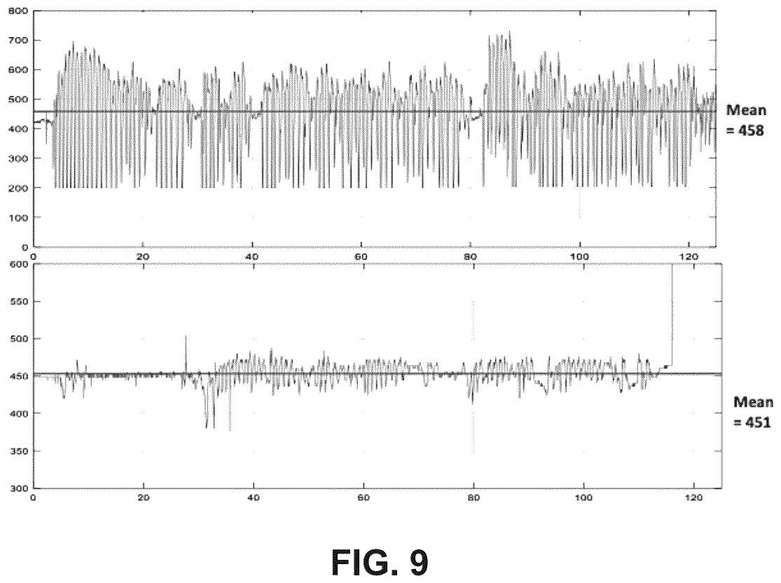

FIG. 5 depicts a chart showcasing results of a peak detection method, FIG. 6 depicts two graphs showing detected peaks, FIG. 7 depicts another graph showing detected peaks, FIG. 8 depicts a graph showing an inverted signal and detected troughs, and FIG. 9 depicts two graphs showing raw data with a mean.

As described herein as an example, the peak detection method applies a moving mean of 30 data points to raw data. The MATLAB function “findpeaks” is used to find all local maxima. Peaks less than 20 mmHg above the local mean are filtered out. Moreover, peaks occurring within 0.25 secs of any previous peak are also filtered out.

The graph of FIG. 5 includes a first column 146 associated with a data set number, a second column 148 associated with peak detection (e.g., a number of sucks detected), and a third column 150 associated with Δ. The graphs in FIG. 6, FIG. 7, and FIG. 8 include an x-axis 152 associated with a time in seconds and a y-axis 154 associated with pressure. Sharp peaks 156 are shown in FIG. 7 before a measured suck. FIG. 8 shows a graph where the signal is inverted and troughs are detected instead of peaks. The same results are shown in FIG. 8 as in previous figures. The data in the first graph in FIG. 9 is more symmetric about the mean, as in the peaks and troughs have a similar magnitude relative to the mean. The data in the second graph in FIG. 9 shows the peaks above the mean being wider and shorter, as compared to the troughs.

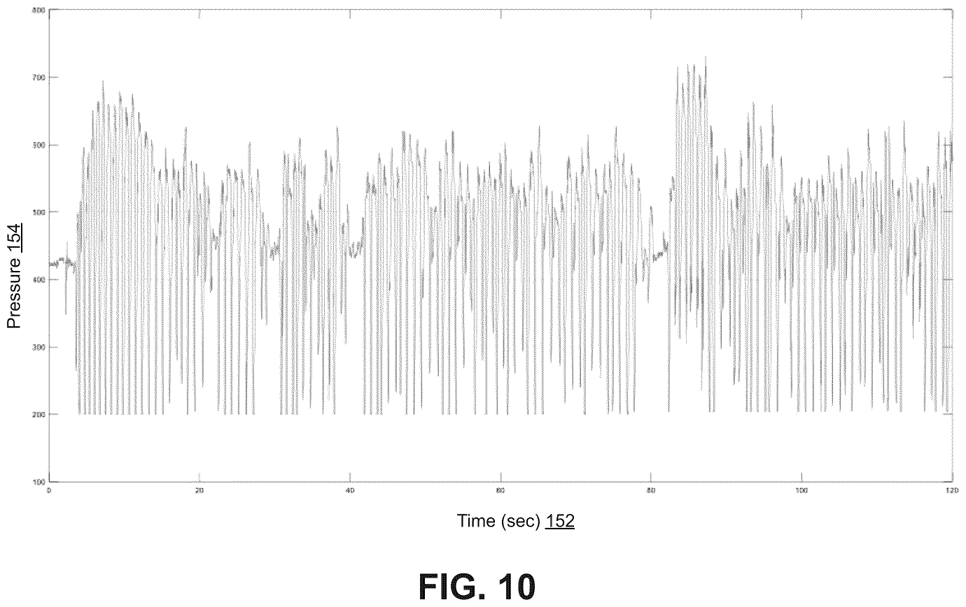

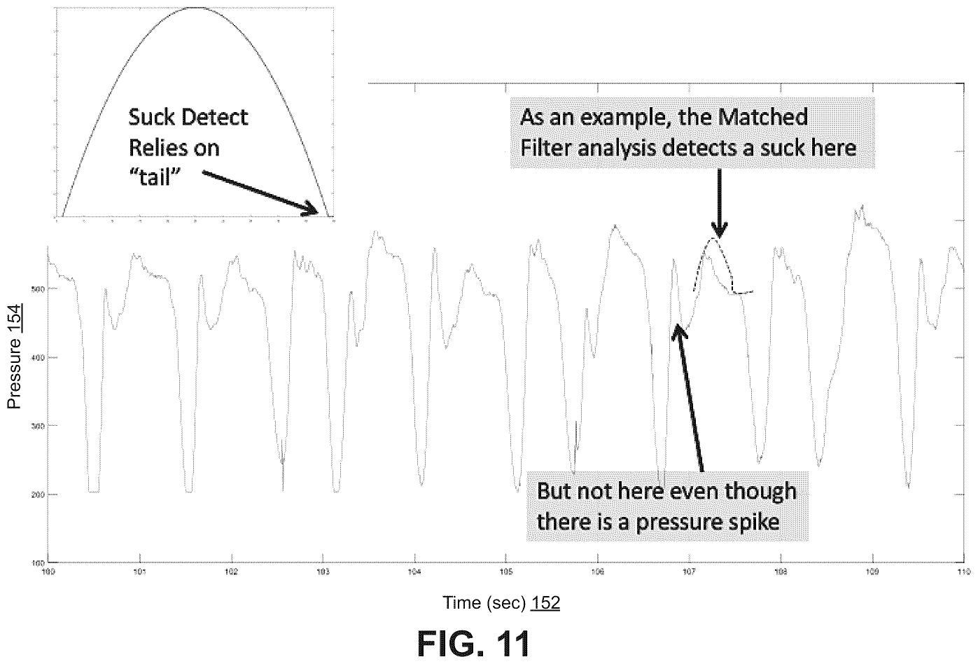

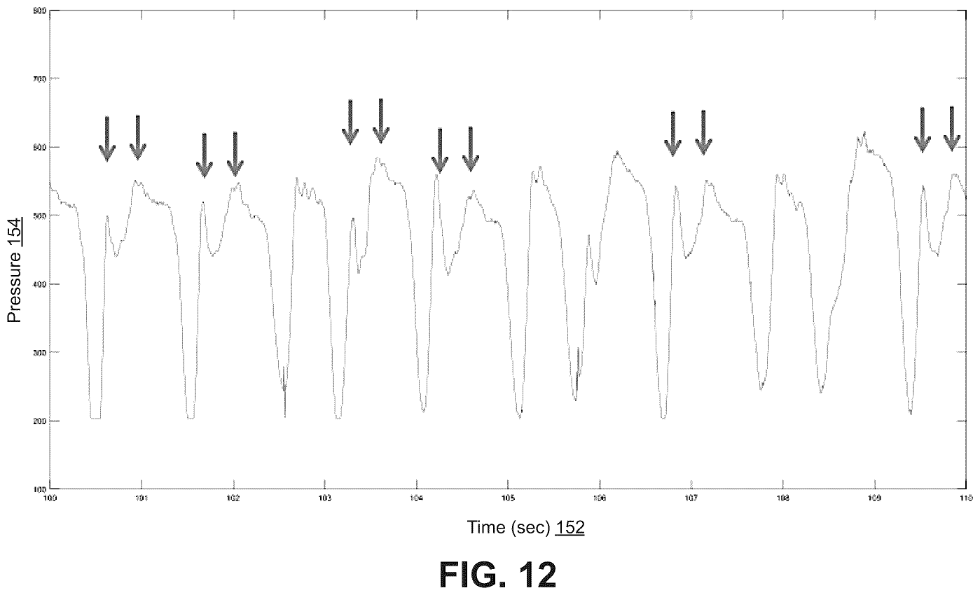

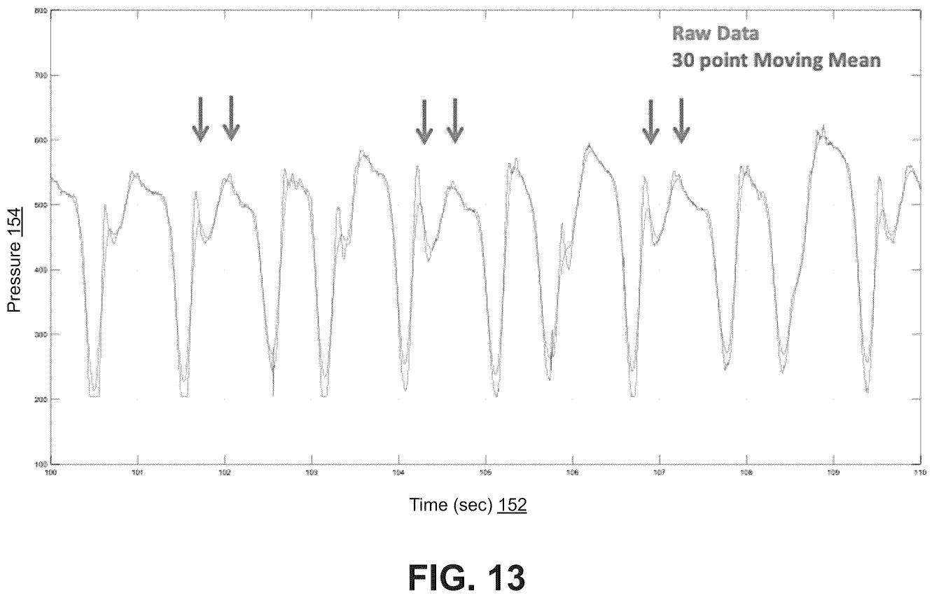

FIG. 10, FIG. 12, and FIG. 13 depict graphs of averaging on peak detection. FIG. 11 depicts a graph utilizing matched filter analysis, according to at least some embodiments disclosed herein. Specifically, each of FIG. 10-FIG. 13 include an x-axis 152 associated with a time in seconds and a y-axis 154 associated with pressure. Raw data is shown in FIG. 10. A matched filter, as discussed in relation to FIG. 11, is obtained by correlating a known delayed signal, or template, with an unknown signal to detect the presence of the template in the unknown signal. The matched filter is the optimal linear filter for maximizing the signal-to-noise ratio (SNR) in the presence of additive stochastic noise. FIG. 12 depicts an example of a “double peak” and with increased smoothing via moving the mean, the double peaks of FIG. 12 are removed in FIG. 13.

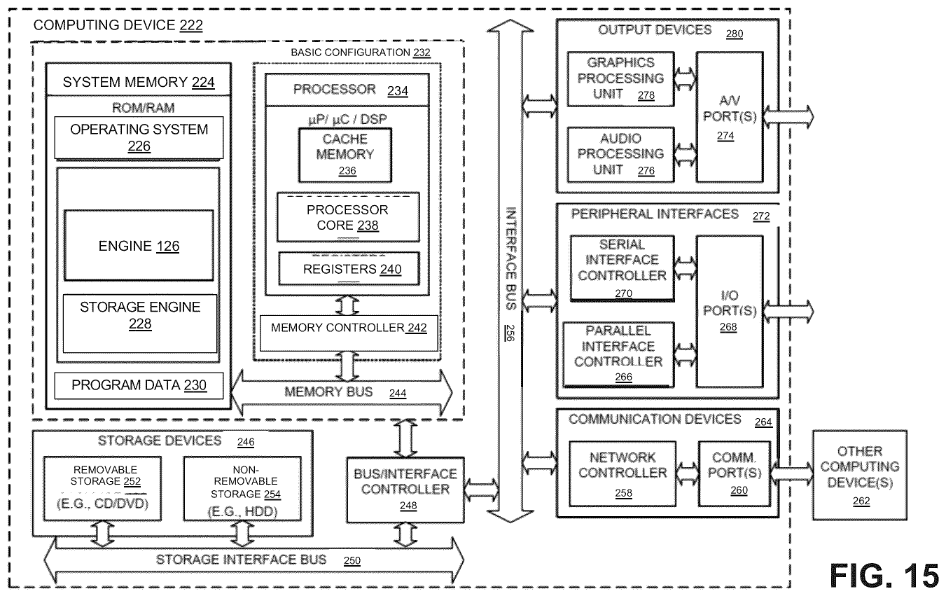

FIG. 15 is a block diagram of a computing device included within the computer system of FIG. 3A and/or FIG. 3C. In some embodiments, the present invention may include a computer system, a method, and/or a computing device 222 (of FIG. 15). A basic configuration 232 of a computing device 222 is illustrated in FIG. 15 by those components within the inner dashed line. In the basic configuration 232 of the computing device 222, the computing device 222 includes a processor 234 and a system memory 224. In some examples, the computing device 222 may include one or more processors and the system memory 224. A memory bus 244 is used for communicating between the one or more processors 234 and the system memory 224.

Depending on the desired configuration, the processor 234 may be of any type, including, but not limited to, a microprocessor (μP), a microcontroller (μC), and a digital signal processor (DSP), or any combination thereof. Further, the processor 234 may include one more levels of caching, such as a level cache memory 236, a processor core 238, and registers 240, among other examples. The processor core 238 may include an arithmetic logic unit (ALU), a floating point unit (FPU), and/or a digital signal processing core (DSP Core), or any combination thereof. A memory controller 242 may be used with the processor 234, or, in some implementations, the memory controller 242 may be an internal part of the memory controller 242.

Depending on the desired configuration, the system memory 224 may be of any type, including, but not limited to, volatile memory (such as RAM), and/or non-volatile memory (such as ROM, flash memory, etc.), or any combination thereof. The system memory 224 includes an operating system 226, one or more engines, such as the engine 126, and program data 230. The system memory 224 may also include a storage engine 228 that may store any information disclosed herein.

Moreover, the computing device 222 may have additional features or functionality, and additional interfaces to facilitate communications between the basic configuration 232 and any desired devices and interfaces. For example, a bus/interface controller 248 is used to facilitate communications between the basic configuration 232 and data storage devices 246 via a storage interface bus 250. The data storage devices 246 may be one or more removable storage devices 252, one or more non-removable storage devices 254, or a combination thereof. Examples of the one or more removable storage devices 252 and the one or more non-removable storage devices 254 include magnetic disk devices (such as flexible disk drives and hard-disk drives (HDD)), optical disk drives (such as compact disk (CD) drives or digital versatile disk (DVD) drives), solid state drives (SSD), and tape drives, among others.

In some embodiments, an interface bus 256 facilitates communication from various interface devices (e.g., one or more output devices 280, one or more peripheral interfaces 272, and one or more communication devices 264) to the basic configuration 232 via the bus/interface controller 256. Some of the one or more output devices 280 include a graphics processing unit 278 and an audio processing unit 276, which are configured to communicate to various external devices, such as a display or speakers, via one or more A/V ports 274.

The one or more peripheral interfaces 272 may include a serial interface controller 270 or a parallel interface controller 266, which are configured to communicate with external devices, such as input devices (e.g., a keyboard, a mouse, a pen, a voice input device, or a touch input device, etc.) or other peripheral devices (e.g., a printer or a scanner, etc.) via one or more I/O ports 268.

Further, the one or more communication devices 264 may include a network controller 258, which is arranged to facilitate communication with one or more other computing devices 262 over a network communication link via one or more communication ports 260. The one or more other computing devices 262 include servers, the database, mobile devices, and comparable devices.

The network communication link is an example of a communication media. The communication media are typically embodied by the computer-readable instructions, data structures, program modules, or other data in a modulated data signal, such as a carrier wave or other transport mechanism, and include any information delivery media. A “modulated data signal” is a signal that has one or more of its characteristics set or changed in such a manner as to encode information in the signal. By way of example, and not limitation, the communication media may include wired media (such as a wired network or direct-wired connection) and wireless media (such as acoustic, radio frequency (RF), microwave, infrared (IR), and other wireless media). The term “computer-readable media,” as used herein, includes both storage media and communication media.

It should be appreciated that the system memory 224, the one or more removable storage devices 252, and the one or more non-removable storage devices 254 are examples of the computer-readable storage media. The computer-readable storage media is a tangible device that can retain and store instructions (e.g., program code) for use by an instruction execution device (e.g., the computing device 222). Any such, computer storage media is part of the computing device 222.

The computer readable storage media/medium can be a tangible device that can retain and store instructions for use by an instruction execution device. The computer readable storage media/medium may be, for example, but is not limited to, an electronic storage device, a magnetic storage device, an optical storage device, an electromagnetic storage device, and/or a semiconductor storage device, or any suitable combination of the foregoing. A non-exhaustive list of more specific examples of the computer readable storage media/medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, and/or a mechanically encoded device (such as punch-cards or raised structures in a groove having instructions recorded thereon), and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Aspects of the present invention are described herein regarding illustrations and/or block diagrams of methods, computer systems, and computing devices according to embodiments of the invention. It will be understood that each block in the block diagrams, and combinations of the blocks, can be implemented by the computer-readable instructions (e.g., the program code).

The computer-readable instructions are provided to the processor 234 of a general purpose computer, special purpose computer, or other programmable data processing apparatus (e.g., the computing device 222) to produce a machine, such that the instructions, which execute via the processor 234 of the computer or other programmable data processing apparatus, create means for implementing the functions/acts specified in the block diagram blocks. These computer-readable instructions are also stored in a computer-readable storage medium that can direct a computer, a programmable data processing apparatus, and/or other devices to function in a particular manner, such that the computer-readable storage medium having instructions stored therein comprises an article of manufacture including instructions, which implement aspects of the functions/acts specified in the block diagram blocks.

The computer-readable instructions (e.g., the program code) are also loaded onto a computer (e.g. the computing device 222), another programmable data processing apparatus, or another device to cause a series of operational steps to be performed on the computer, the other programmable apparatus, or the other device to produce a computer implemented process, such that the instructions, which execute on the computer, the other programmable apparatus, or the other device, implement the functions/acts specified in the block diagram blocks.

Computer readable program instructions described herein can also be downloaded to respective computing/processing devices from a computer readable storage medium or to an external computer or external storage device via a network (e.g., the Internet, a local area network, a wide area network, and/or a wireless network). The network may comprise copper transmission cables, optical transmission fibers, wireless transmission, routers, firewalls, switches, gateway computers, and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer/computing device, partly on the user's computer/computing device, as a stand-alone software package, partly on the user's computer/computing device and partly on a remote computer/computing device or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to block diagrams of methods, computer systems, and computing devices according to embodiments of the invention. It will be understood that each block and combinations of blocks in the diagrams, can be implemented by the computer readable program instructions.

The block diagrams in the Figures illustrate the architecture, functionality, and operation of possible implementations of computer systems, methods, and computing devices according to various embodiments of the present invention. In this regard, each block in the block diagrams may represent a module, a segment, or a portion of executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block and combinations of blocks can be implemented by special purpose hardware-based systems that perform the specified functions or acts or carry out combinations of special purpose hardware and computer instructions.

### EXAMPLES

### Example 1

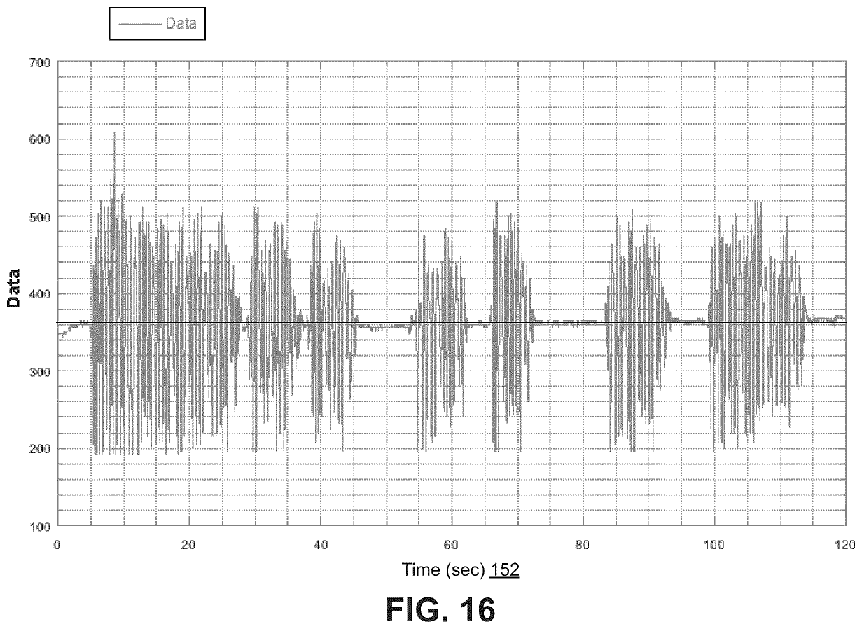

As an example, a study of 124 neonates, or infants, was carried out using the device 100. Out of these 124 neonates, 40 neonates were born to mothers on methadone and 28 neonates were premature at their release from the hospital. FIG. 16 includes an x-axis 152 measuring a time in seconds and depicts a two minute plot of data from a “normal” neonate showing a strong nipple bite causing a cut-off at 200. The broad behavior of this suck-swallow feeding trace (SSFT) shows a large burst of sucks and swallows at the beginning of the feeding followed by shorter bursts.

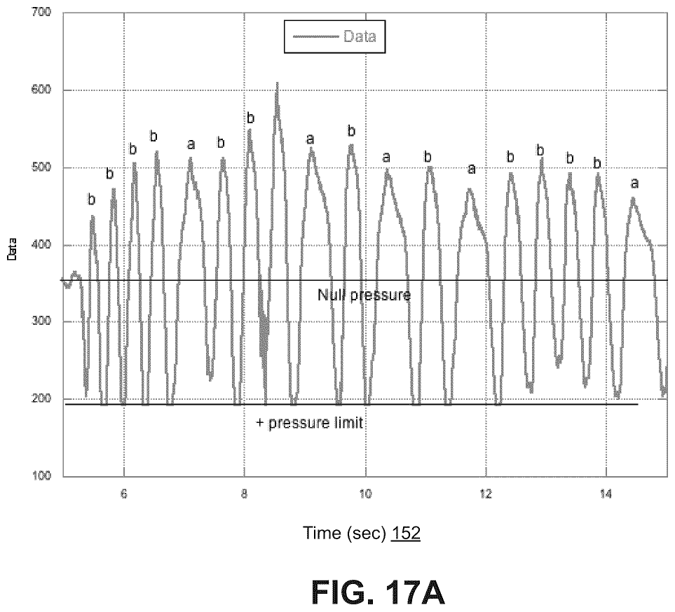

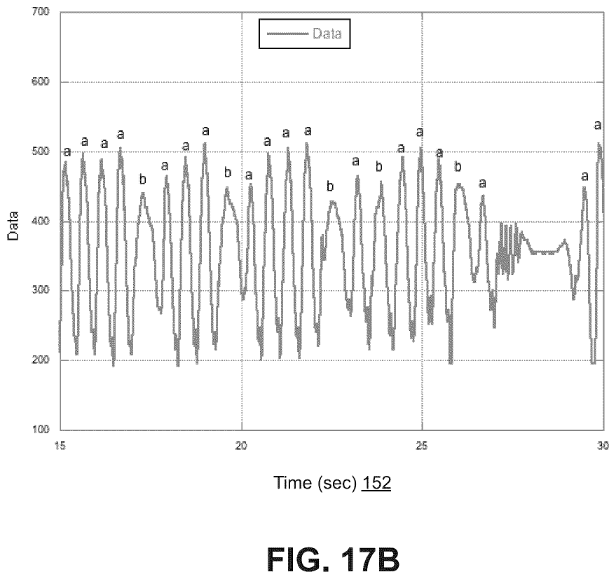

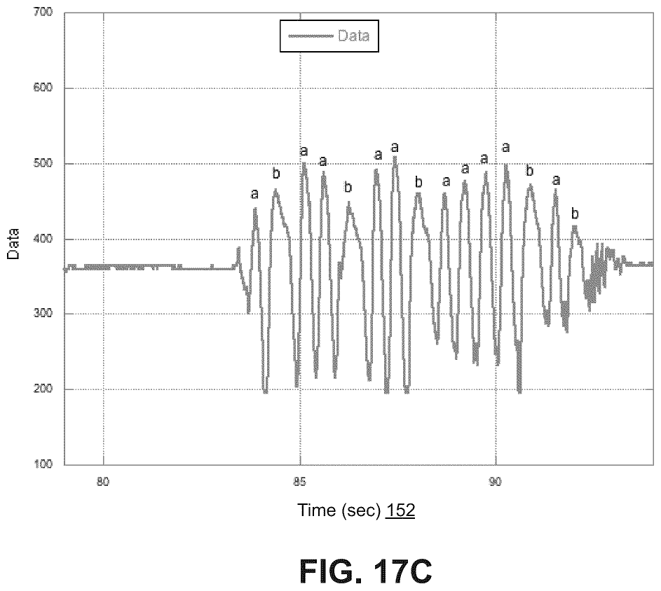

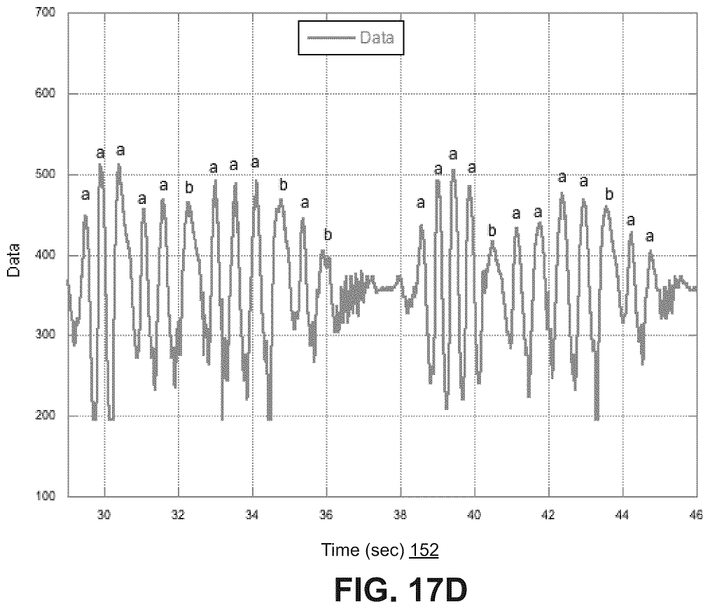

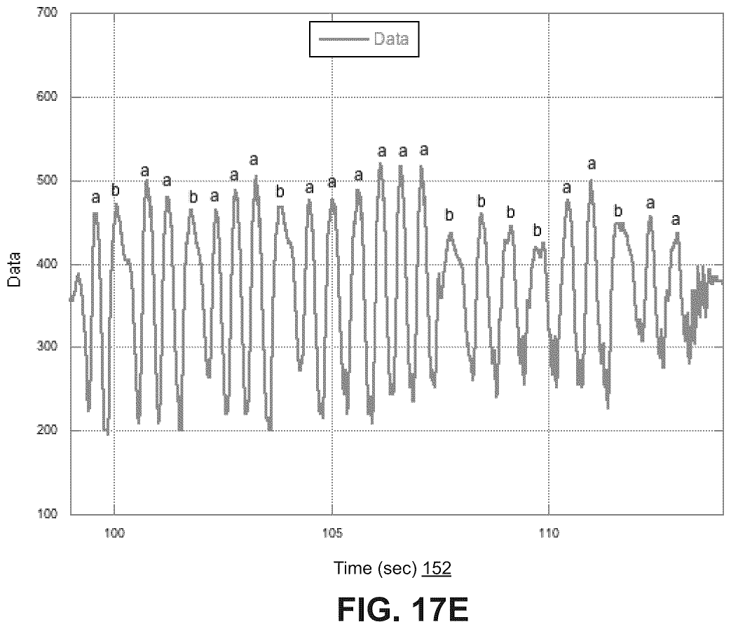

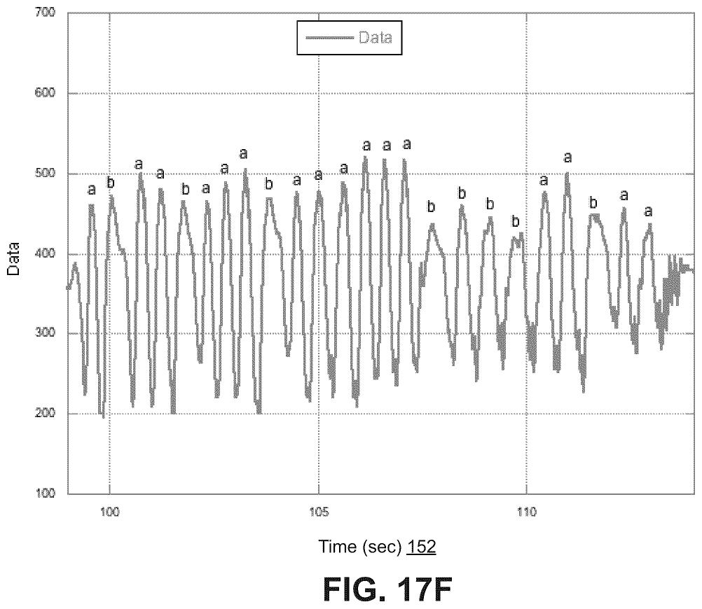

The data of FIG. 16 is expanded to better examine the pattern in the SSFT in FIG. 17A-FIG. 17F. FIG. 17A-FIG. 17F also include the x-axis 152. Specifically, the x-axis 152 in FIG. 17A-FIG. 17F measures 15 seconds. The maxima marked “a” are assumed to be sucks and the peaks marked “b” are assumed to involve breathing. FIG. 17A-FIG. 17F also include a horizontal line at approximately a 345 level, which is a neutral (atmospheric pressure) point reading generated by the pressure sensor 122. Negative (suck) pressure is shown above this horizontal line.

Since the data associated with the neonates in FIG. 17A-FIG. 17F were not exposed to opiates during their gestation, the following two assumptions were made: (1) there should be significant similarity in the signal trace during the first 20-30 seconds of feeding and (2) there should be significant similarity with a 20-30 second interval toward the end of the 120 second measurement.

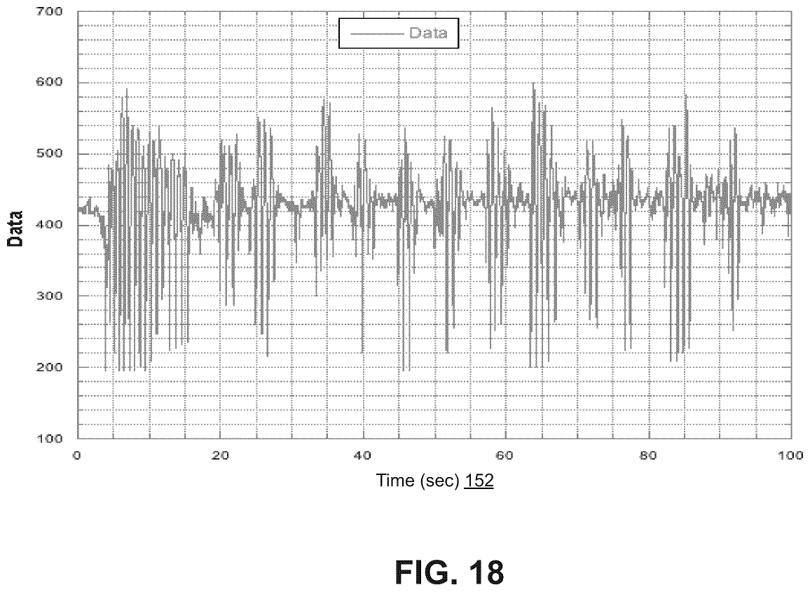

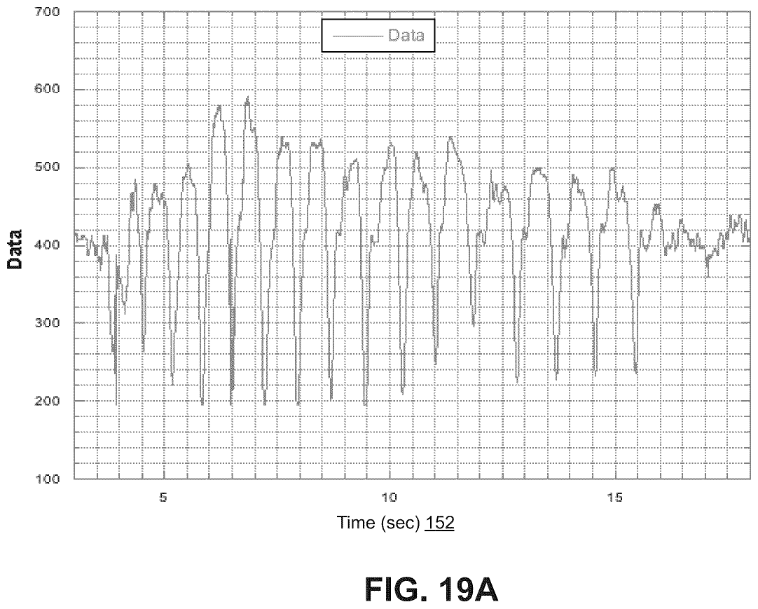

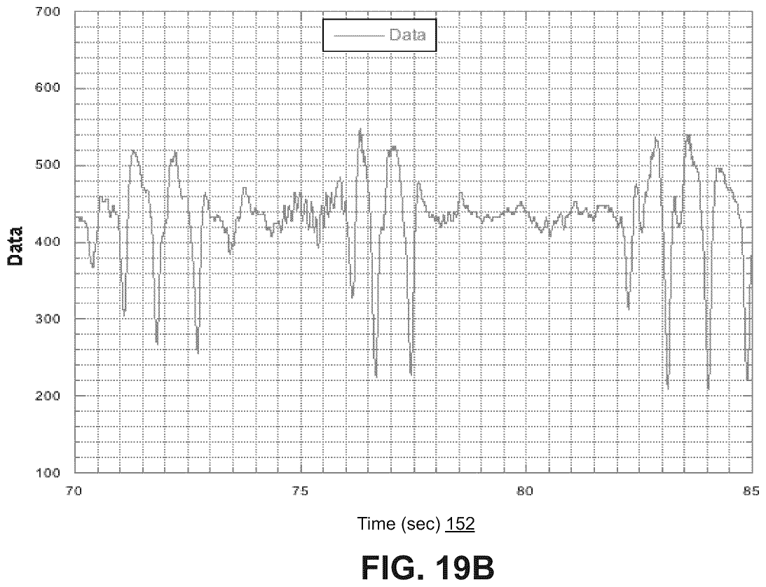

Data associated with another neonate is shown in FIG. 18. The data of FIG. 18 is expanded in FIG. 19A and FIG. 19B. In FIG. 19A and FIG. 19B, the SSFT segments are 15 seconds long. The neonate in FIG. 19A and FIG. 19B is a slower feeder than the neonate in FIG. 17A-FIG. 17F. In FIG. 17A-FIG. 17F, the neonate had 9 sucks in 4-5 seconds while the neonate in FIG. 19A and FIG. 19B had about 1 suck per second. In addition, the shape of the sucks was different. Instead of peaking initially as in the “b” sucks of FIG. 17A-FIG. 17F, there was a shoulder before many of the sucks in FIG. 19A and FIG. 19B.

This data showed that each neonate had a more or less characteristic pattern of individual suck/swallow, but all of the neonates displayed uniform and reproducible patterns. Further, this data showed the variations in the shape of an individual suck and an individual swallow.

### Example 2

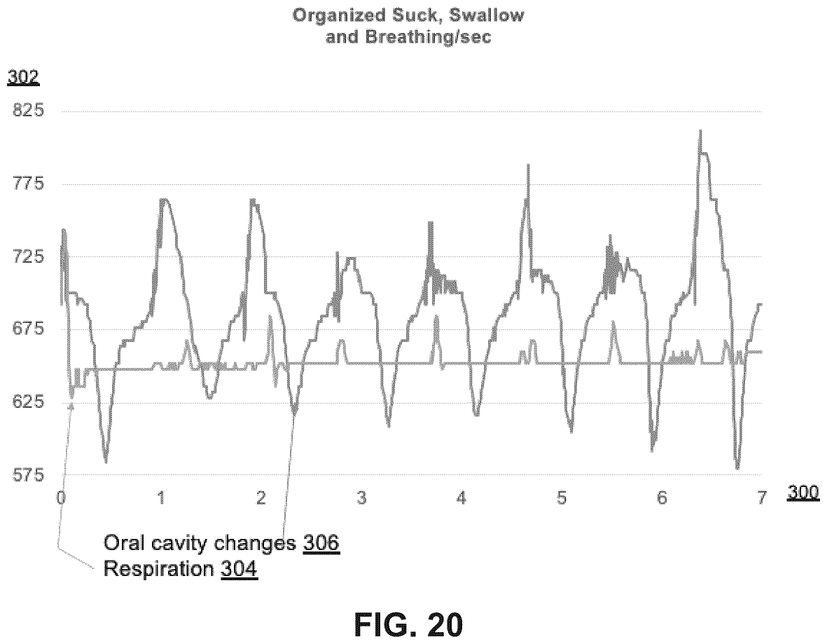

At least three interface tools are available to help guide clinical decision making. Specifically, FIG. 20 depicts a graphical qualitative representation of a feed from the device 100 illustrating organized suck, swallow, and breathing per second, according to at least some embodiments disclosed herein. Specifically, FIG. 20 includes an x-axis 300 and a y-axis 302. FIG. 20 graphs both oral cavity changes 306 and respiration 304 measured by the device 100.

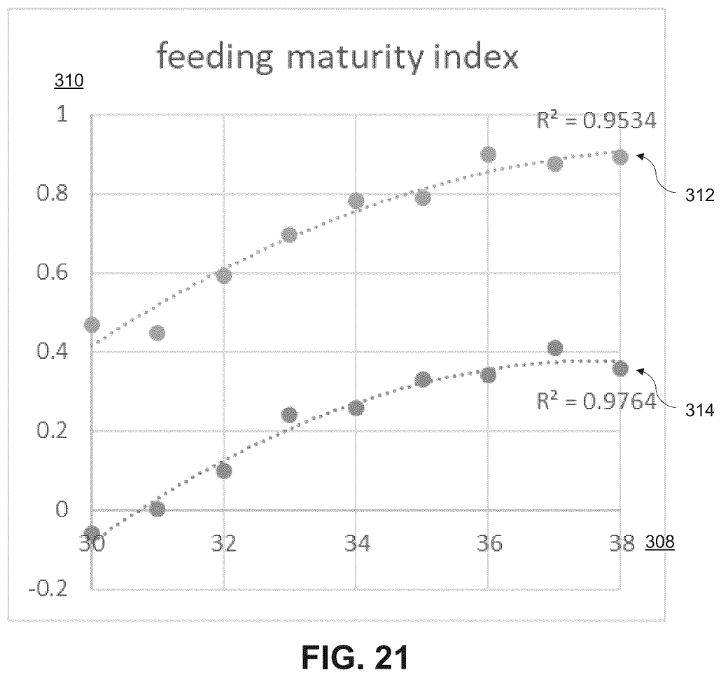

Moreover, FIG. 21 depicts a graph illustrating a feeding maturity curve, according to at least some embodiments disclosed herein. Specifically, FIG. 21 includes an x-axis 308 and a y-axis 310. FIG. 21 illustrates a first measurement 312 with an R² value of 0.9534 and a second measurement 314 with an R² value of 0.9764.

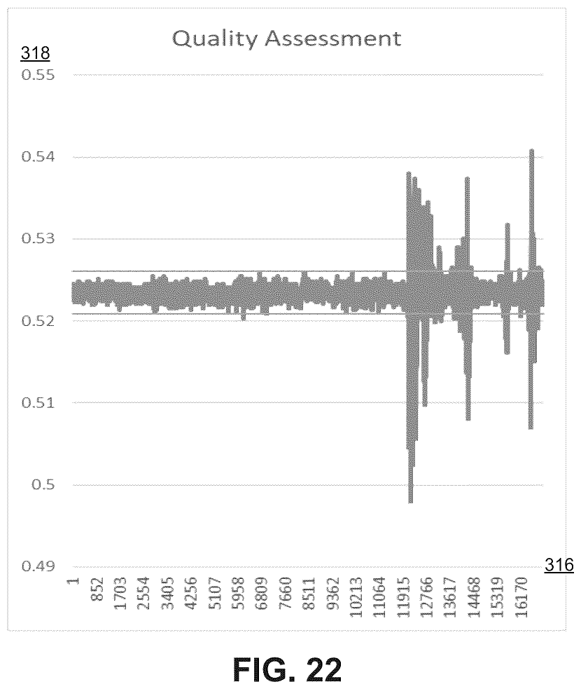

FIG. 22 depicts a graph illustrating quality assessment curve/analysis, according to at least some embodiments disclosed herein. FIG. 22 includes an x-axis 316 and a y-axis 318. Specifically, FIG. 22 depicts time to decay in feeding performance, as well as a percentage within expected variations.

### Example 3

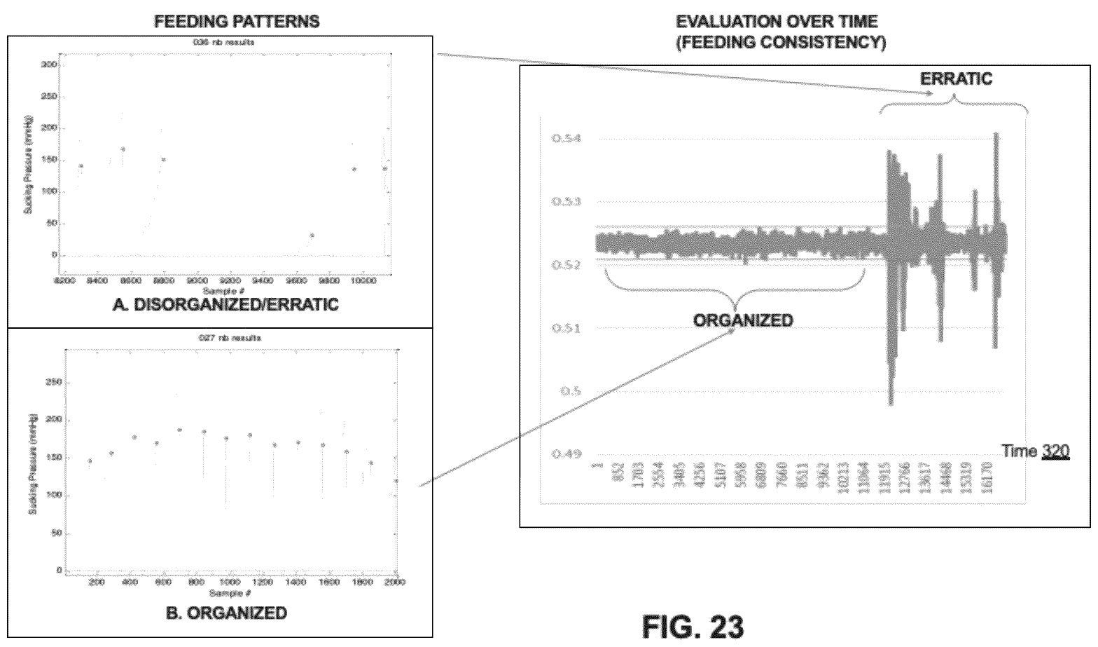

FIG. 23 depicts a first graph A illustrating disorganized/erratic feeding patterns, a second graph B illustrating organized feeding patterns, and a third graph illustrating evaluation over time or feeding consistency, according to at least some embodiments disclosed herein. Specifically, the third graph includes an x-axis 320 associated with time and depicts both erratic and disorganized/erratic feeding patterns. As such, the device 100 assesses real-time surgical recovery status by monitoring duration of organized feeding skills.

### Example 4

As described herein, neonatal abstinence syndrome (NAS) is a group of conditions caused when a baby withdraws from certain drugs he/she is exposed to in the womb before birth. NAS is most often caused when a woman takes opioids during pregnancy. These infants have problems feeding.

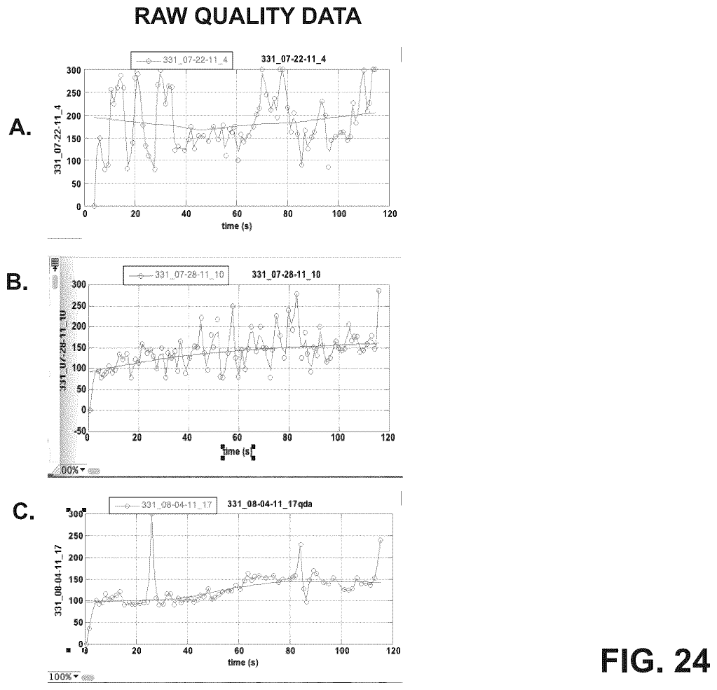

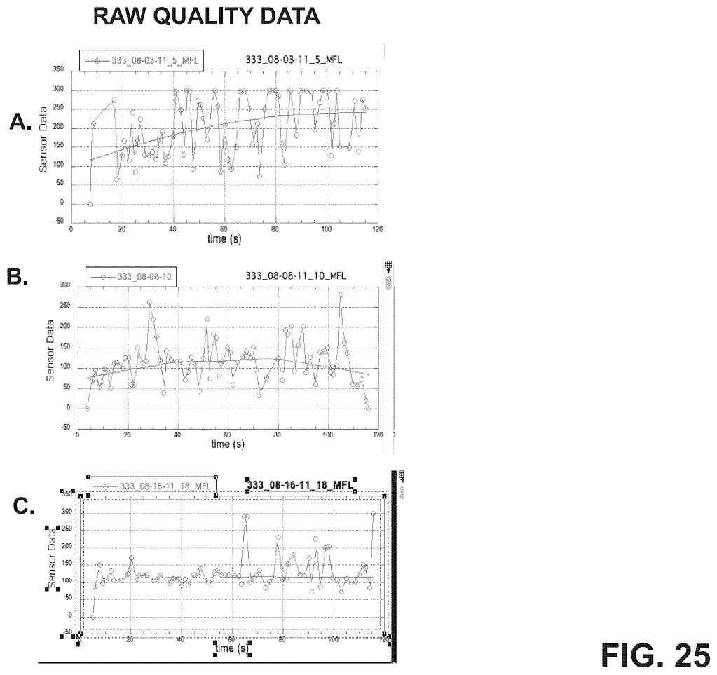

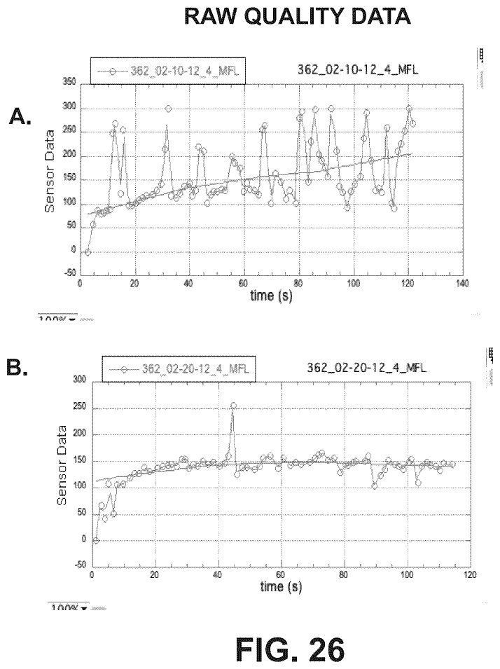

FIG. 24 depicts three graphs illustrating examples of neonatal abstinence syndrome (NAS) recovery for a first patient, FIG. 25 depicts an additional three graphs illustrating examples of NAS recovery for a second patient, and FIG. 26 depicts an additional two graphs illustrating examples of NAS recovery for a third patient, according to at least some embodiments disclosed herein.

The graphs in FIG. 24-FIG. 26 look at frequency consistency over the duration of a feeding. The sequential graphs (e.g., graph A, graph B, and graph C of the respective figures) represent the infant “recovering” from their addiction. The same changes or consistency changes may be seen if the infant is recovering in surgery.

### Example 5

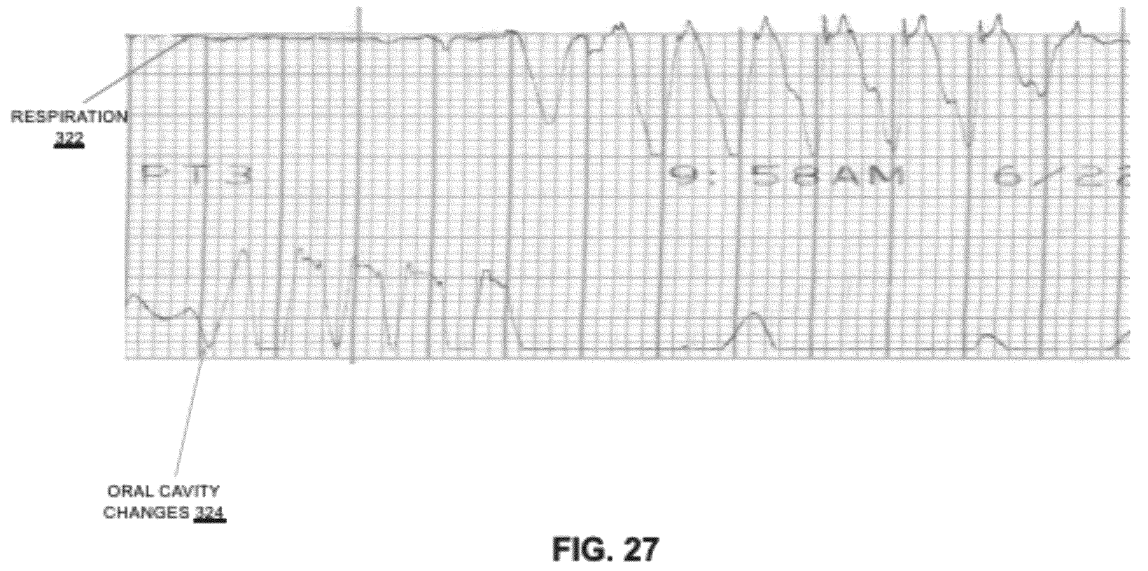

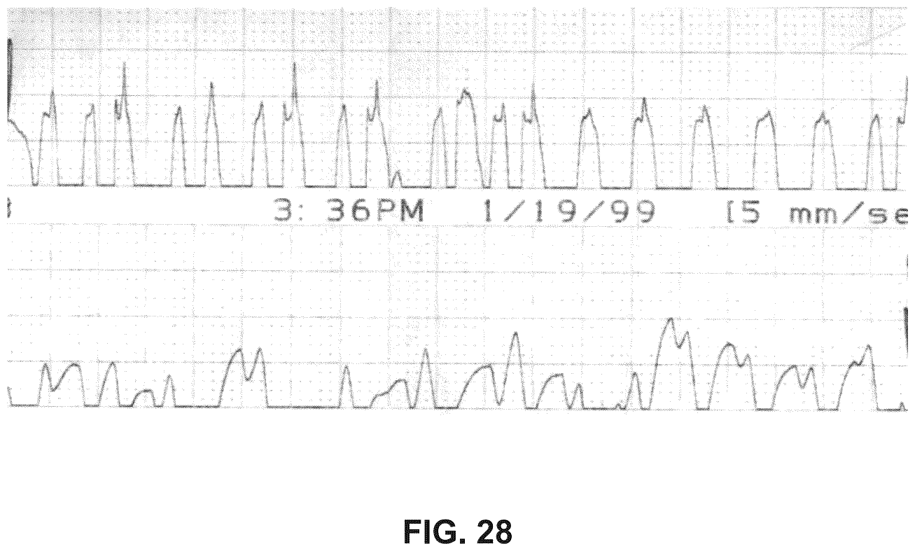

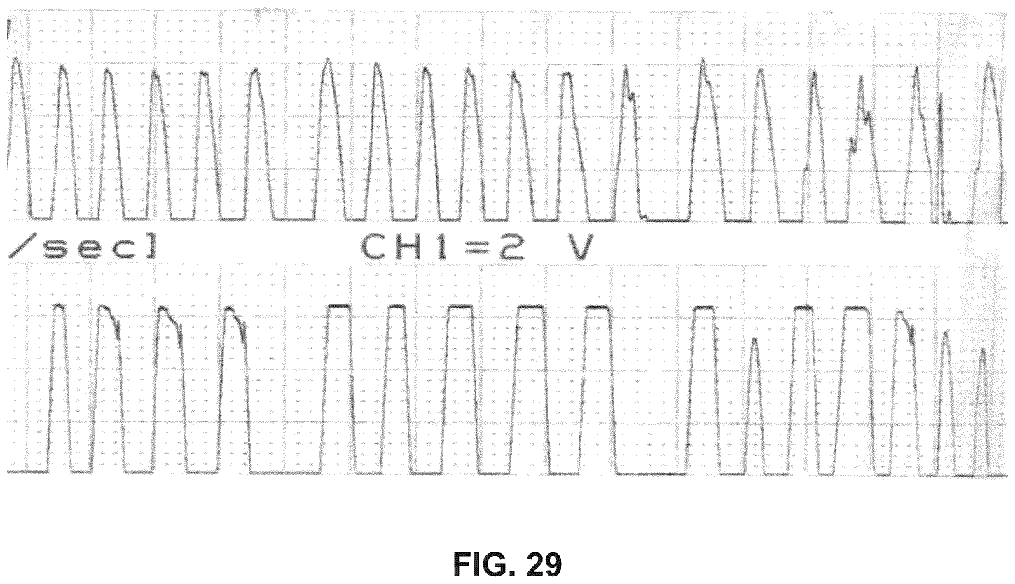

As described herein, a developmental biomarker was created based on the analysis of five specific characteristics. Specifically, FIG. 27 depicts a graph illustrating respiration and oral cavity changes associated with immature feeding, FIG. 28 depicts a graph illustrating developing feeding and FIG. 29 depicts a graph illustrating mature feeding, according to at least some embodiments disclosed herein. Specifically, the top image in FIG. 28 and FIG. 29 depicts respiration and the bottom image in FIG. 28 and FIG. 29 depicts oral cavity pressure changes, showing the suck. Moreover, respiration 322 and oral cavity changes 324 are depicted in FIG. 27.

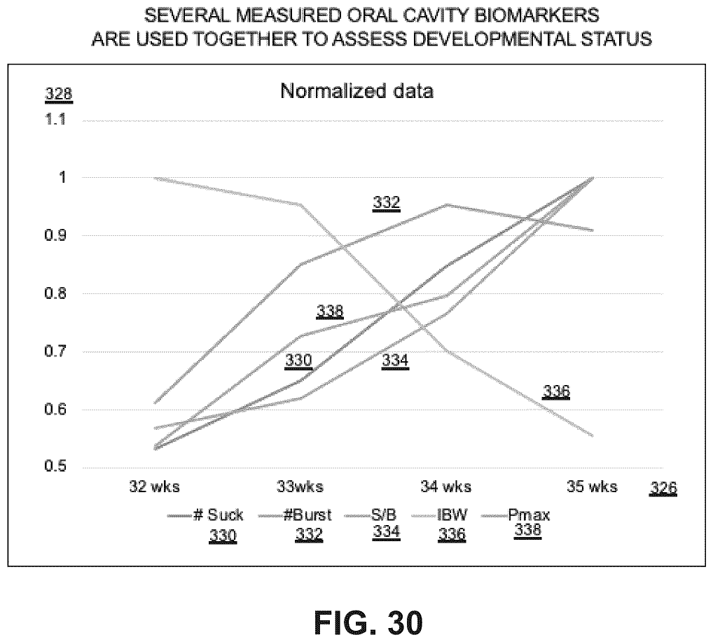

FIG. 30 depicts a graph illustrating several measured oral cavity biomarkers that are used together to assess a developmental status for an infant, according to at least some embodiments disclosed herein. Specifically, FIG. 30 includes an x-axis 326 associated with a number of weeks and a y-axis 328. The data in FIG. 30 is normalized and includes a number of sucks 330, a number of bursts 332, S/B 334, IBW 336, and Pmax 338.

### Example 6

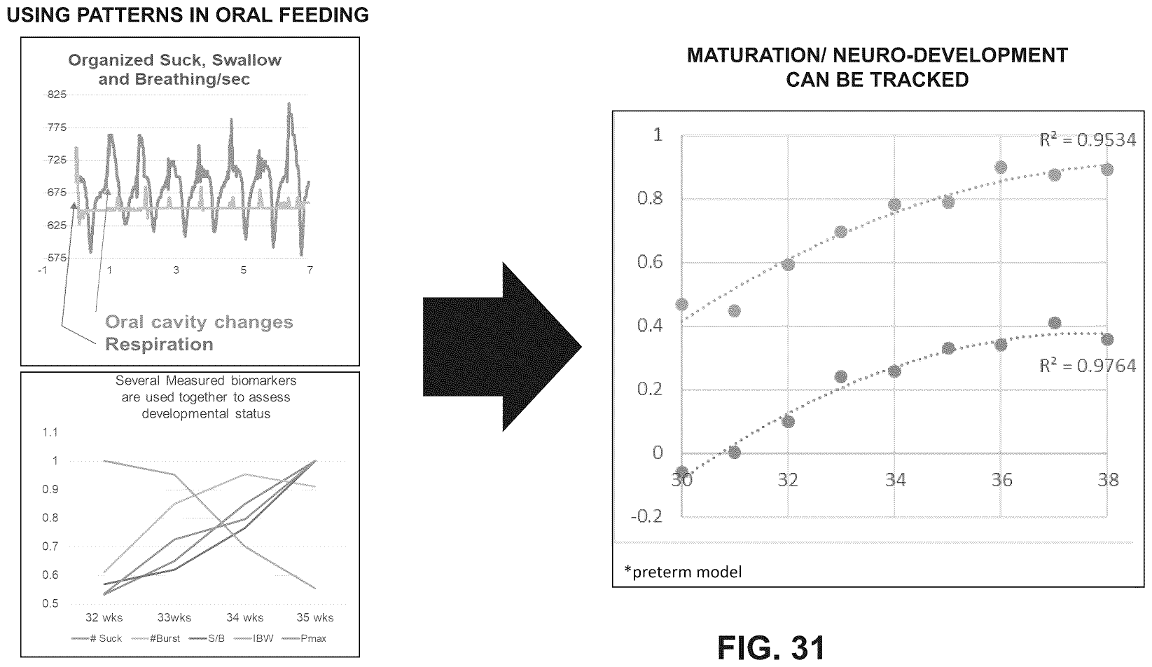

As described herein, neurological maturation biomarkers are used to assess infant feeding progress. Specifically, FIG. 31 depicts graphs illustrating using patterns in oral feeding and the tracking of maturation/neuro-development, according to at least some embodiments disclosed herein. Specifically, as shown in FIG. 31, a first graph depicts organized suck, swallow, and breathing/second (e.g., FIG. 20) and a second graph depicts several measured biomarkers used together to assess an infant's developmental status (e.g., FIG. 30). These two graphs may be used to track maturation/neuro-development of the infant, as shown in a third graph (e.g., FIG. 21).

### Example 7

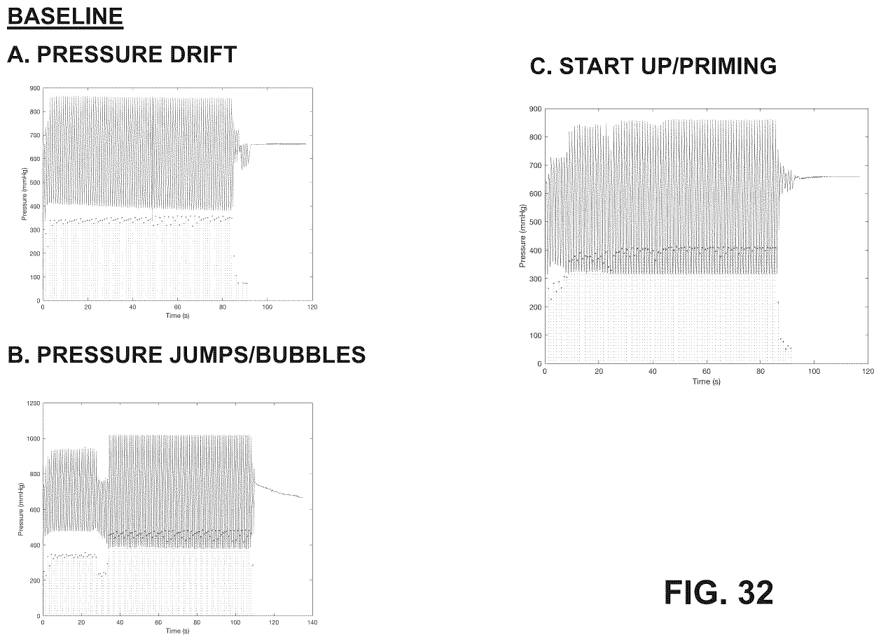

FIG. 32 depicts graphs showing variables on a bench that need to be managed, according to at least some embodiments disclosed herein. Specifically, FIG. 32 depicts a first graph showing baseline pressure drift, a second graph showing baseline pressure jumps/bubbles, and a third graph showing start-up/priming. For a baseline pressure measurement, an amplitude of suck, swallow and respiration is needed. The alignment of the suck/swallow data to the respiratory data allows for the extraction of simultaneous features and for the development of multiple sucks in a single swallow.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others or ordinary skill in the art to understand the embodiments disclosed herein.

When introducing elements of the present disclosure or the embodiments thereof, the articles “a,” “an,” and “the” are intended to mean that there are one or more of the elements. Similarly, the adjective “another,” when used to introduce an element, is intended to mean one or more elements. The terms “including” and “having” are intended to be inclusive such that there may be additional elements other than the listed elements.

Although this invention has been described with a certain degree of particularity, it is to be understood that the present disclosure has been made only by way of illustration and that numerous changes in the details of construction and arrangement of parts may be resorted to without departing from the spirit and the scope of the invention.

### REFERENCES

The following references, to the extent that they provide details supplementary to those set forth herein, are specifically incorporated herein by reference.
Abu-Shaweesh, J. (2004). Maturation of Respiratory Relfex Responses in the Fetus and Neonate. Seminars in Neonatology. DOI: 10.1016/j.siny.2003.09.003.
Adams-Chapman, I., et al. (2013). Association Between Feeding Difficulties and Language Delay in Preterm Infants Using Bayley Scals of Infant Development—Third Edition. J. Pediatr. DOI: 10.1016/j.jpeds.2013.03.006.
Barlow, S. (2009). Oral and Respiratory Control for Preterm. Feeding Current Opinion in Otolarnygollogy & Head and Neck Surgery. DOI: 10.1097/MOO.0b013e32832b36fe.
Capilouto, G., et al. (2016). Objective Assessment of a Preterm Infant's Nutritive Sucking From Initiation of Feeding Through Hospitalization and Discharge. Neonatal Intensive Care. PMID: 28008218.
Carty, C. L., et al. (2018). The Giving Parents Support Study: A Randomized Clinical Trial of a Parent Navigator Intervention to Improve Outcomes After Neonatal Intensive Care Unit Discharge. Contemporary Clinial Trials. DOI: 10.1016/j.cct.2018.05.004.
Cerro, N., et al. (2002). Eating Behaviour of Children 1.5-3.5 Years Born Preterm: Parents' Perceptions. J. Paediatr Child Health. DOI: 10.1046/j.1440-1754.2002.00728.x.
Craig, C. M., et al. (1999). Neonatal Control of Nutritive Sucking Pressure: Evidence for an Intrinsic Tau-Guide. Exp Brain Res. DOI: 10.1007/s002210050634.
Daily, W., et al. (1968). Apnea in Premature Infants: Monitoring, Incidences, Heart Rate Changes, and an Effect on Environmental Temperature. Pediatrics. PMID: 5777065.
Forchielli, M., et al. (1994). Children with Congenital Heart Disease: A Nutritional Challenge. Nutrition Reviews. DOI: 10.1111/j.1753-4887.1994.tb01359.x
Gewolb, I., et al. (2001). Developmental Patterns of Rhythmic Suck and Swallow in Preterm Infants. Dev. Med. Child. Neurol. DOI: 10.1017/s0012162201000044.
Gewolb, I., et al. (2006). Maturational Changes in the Rhythms, Patterning and Coordination of Respiration and Swallow During Preterm and Term Infants. Dev. Med Child Neurol. DOI: 10.1017/S001216220600123X.
Gewolb, I. H., et al. (2004). Coordination of Suck-Swallow-Respiration in Infants Born to Mothers with Drug-Abuse Problems. Dev. Med. Child Neurol. PMID: 15473175.
Hartman, D., et al. (2012). Transition to Home After Neonatal Surgery for Congenital Heart Disease. MCN Am. J. Matern. Child. Nurs. DOI: 10.1097/NMC.0b013e318241dac1.
Hawdon, J. M., et al. (2000). Identification of Neonates at Risk of Developing Feeding Problems in Infancy. Dev. Med. Child. Neurol. DOI: 10.1017/s0012162200000402.
Hehir, D., et al. (2011). Feeding, Growth, Nutrition, and Optimal Interstage Surveillance for Infants with Hypoplastic Left Heart Syndrome. Cardiology in the Young. DOI: 10.1017/S1047951111001600.
Hiramoto, A., et al. (2014). Abnormal Sucking Behavior in Infants as a Predictor of Developmental Delay in 18 Month or 3 Year of Age. Journal of Brain Science. DOI: 10.20821/jbs.43.0_5.
Huysman, W.A., et al. (2003). Growth and Body Composition in Preterm Infants with Bronchopulmonary Dysplasia. Arch Dis Child Fetal Neonatal Ed. DOI: 10.1136/fn.88.1.f46.
Jadcherla, S. R., et al. (2009). Leuthner, Feeding Abilities in Neonates with Congenital Heart Disease: A Retrospective Study. J Perinatol. DOI: 10.1038/jp.2008.136.
Lau, C., et al. (2000). Characterization of the Developmental Stages of Sucking in Preterm Infants During Bottle Feeding. Acta Paediatr. PMID: 10943969.
Law-Morstatt, et al. (2003). Pacing as a Treatment Technique for Transitional Sucking Patterns. Journal of Perinatology. DOI: 10.1038/sj.jp.7210976.
Medoff-Cooper, B., et al. (2020). Telehealth Home Monitoring and Postcardiac Surgery for Congenital Heart Disease. Pediatrics. DOI: 10.1542/peds.2020-0531.
Medoff-Cooper, B., et al. (2011). Weight change in infants with a functionally univentricular heart: from surgical intervention to hospital discharge. Cardiol. Young. DOI: 10.1017/S104795111000154X.
Medoff-Cooper, B., et al. (2009). Sucking Behavior of Preterm Neonates as a Predictor of Developmental Outcomes. J. Dev. Behav. Pediatr. DOI: 10.1097/DBP.0b013e318196b0a8.
Medoff-Cooper, B., et al. (2002). Feeding Patterns of Full Term and Preterm Infants at Forty Weeks Post-Conceptional Age. J. D. Behav. Pediatr. DOI: 10.1097/00004703-200208000-00007.
Medoff-Cooper, B., et al. (2001). Suckling Behavior as a Function of Gestational Age: A Cross-Sectional Study. Infant Behavior and Development. DOI: 10.1016/S0163-6383(01)00063-7.
Medoff-Cooper, B., et al. (2000). Nutritive Sucking and Neurobehavioral Development in Preterm Infants from 34 Weeks PCA to Term. MCN Am. J. Matern. Child. Nurs. DOI: 10.1097/00005721-200003000-00004.
Miller, M., et al. (2004). Relationship Between Respiratory Control and Feeding in the Developing Infant. Seminars in Neonatology. DOI: 10.1016/j.siny.2003.11.006.
Mussatto, K.A., et al. (2014). Risk and Prevalence of Developmental Delay in Young Children With Congenital Heart Disease. Pediatrics. DOI: 10.1542/peds.2013-2309.
Pickler, R., et al. (2006). Predictors of Nutritive Sucking on Preterm Infants. Journal of Perinatology. DOI: 10.1038/sj.jp.7211590.
Rogers, E. J., et al. (2003). Barriers to Adequate Nutrition in Critically Ill Children. Nutrition. DOI: 10.1016/s0899-9007(03)00170-9.
Siddell, E. P., et al. (1994). A National Survey of Neonatal Intensive-Care Units: Criteria Used to Determine Readiness for Oral Feedings. J Obstet Gynecol Neonatal Nurs. DOI: 10.1111/j.1552-6909.1994.tb01953.x.
Simpson, C., et al. (2002). Early Introduction of Oral Feeding in Preterm Infants. Pediatrics. DOI: 10.1542/peds.110.3.517.
Slattery, J., et al. (2012). Early Sucking and Swallowing Problems as Predictors of Neurodevelopmental Outcome in Children with Neonatal Brain Injury: A Systematic Review. Dev Med Child Neurol. DOI: 10.1111/j.1469-8749.2012.04318.x.
Stevenson, R., et al. (1991). The Development of Normal Feeding and Swallowing. Pediatr. Clin. North Am. DOI: 10.1016/s0031-3955(16)38229-3.
Wolff, P. (1966). Causes, Controls, and Organization of Behaviors in the Neonate. Psychol. Issues. PMID: 5984605.
Wolkhuis-Stigter, M. I., et al. (2016). The Association Between Suckling Behavior in Preterm Infants and Neurodevelopmental Outcomes at 2 Years of Age. J. Pediatrics. DOI: 10.1016/j.jpeds.2014.09.007.

1.-39. (canceled)

40. A method executed by an engine on a computing device for quantitatively measuring infant feeding performance, the method comprising:
analyzing data received from a device affixed to a bottle and associated with a feeding instance of an infant during a first time period;
receiving, from a user and via a graphical user interface (GUI) of the computing device, non-sensor data during the first time period;
determining a baseline pressure from the data received from the device;
detecting sucks by comparing pressure fluctuations to known characteristics;
detecting swallows by comparing pressure fluctuations to known characteristics;
detecting respiration by comparing temperature fluctuations caused by inhalation and exhalation by the infant to known characteristics;
calculating a second time period between the sucks or the swallows to detect bursts;
calculating a third time period without the sucks and without the swallows;
calculating at least one biomarker for the infant or the feeding instance;
analyzing the at least one biomarker to track maturation, neuro-development, or recovery of the infant; and
outputting results of the feeding instance via the GUI to the user.

41. The method of claim 40, further comprising:
detecting characteristic shapes of a specific patient population, a medical condition, or a given biomarker; and/or
adapting the baseline pressure to or within an actual reading.

42. The method of claim 40, further comprising:
utilizing the at least one biomarker to calculate tracking indicators; and
comparing at least one biomarker or the tracking indicators for the infant to determine how they change over time; and/or
comparing the at least one biomarker and the tracking indicators of the infant to a dataset to determine a normative comparison.

43. The method of claim 40, wherein the results comprise curves depicting at least one of feeding trace data, maturation progress data, duration of quality data, and recovery data, and/or wherein:
the feeding trace data comprises at least one of: an average number of sucks/bursts in the first time period, an average length of time between bursts in the first time period, a frequency of sucks in a first two minutes in the first time period, a normal respiratory rate during the first time period, and an overall duration of feed during the first time period; and/or
utilizing the maturation progress data for curve tracking, slope tracking, and to determine a speed of change; and/or
the duration of quality data comprises a score for a quality of the feeding instance during a discrete portion of the feeding instance and a length of time the quality is maintained.

44. The method of claim 40, further comprising:
receiving another set of data from a third-party; and
incorporating the other set of data into the analysis of the infant feeding instance, and/or wherein the other set of data comprises at least one of: a component of an oral feeding evaluation, a clinician evaluation of feeding quality, and an anthropometric evaluation completed prior to discharge and post-discharge.

45. A computer system comprising:
one or more processors;
one or more memories; and
one or more computer-readable hardware storage devices, the one or more computer-readable hardware storage devices containing program code executable by the one or more processors via the one or more memories to implement a method for quantitatively measuring infant feeding performance, the method comprising:
analyzing data received from a device affixed to a bottle and associated with a feeding instance of an infant during a first time period;
receiving, from a user and via a graphical user interface (GUI), non-sensor data during the first time period;
determining a baseline pressure from the data received from the device;
adapting the baseline pressure to an actual reading;
detecting sucks by comparing pressure fluctuations to known characteristics;
detecting swallows by comparing pressure fluctuations to known characteristics;
detecting respiration by comparing temperature fluctuations caused by inhalation and exhalation by the infant to known characteristics;
calculating a second time period between the sucks or the swallows to detect bursts;
calculating a third time period without the sucks and without the swallows;
calculating at least one biomarker for the infant or the feeding instance;
analyzing the at least one biomarker to track maturation and neuro-development of the infant; and
outputting results of the feeding instance via the GUI to the user.

46. A method executed by an engine on a computing device for quantitatively measuring infant feeding performance, the method comprising:
analyzing data received from a device affixed to a bottle and associated with a feeding instance of an infant during a first time period;
receiving, from a user and via a graphical user interface (GUI), non-sensor data during the first time period;
determining a baseline pressure from the data received from the device;
adapting the baseline pressure to an actual reading;
detecting respiration by comparing temperature fluctuations caused by inhalation and exhalation by the infant to known characteristics;
calculating at least one biomarker for the infant or the feeding instance;
analyzing the at least one biomarker to track maturation and neuro-development of the infant; and
outputting results of the feeding instance via the GUI to the user.

47. The method of claim 46, wherein a respiratory measurement component is incorporated into the device and/or wherein the respiratory measurement component fails to come into physical contact with the infant.

48. A device affixed between a nipple and a bottle and comprising embedded electronic components, the embedded electronic components comprising at least one sensor and a computerized data processing system,
the at least one sensor being configured to capture data associated with a feeding instance of an infant during a first time period; and
the computerized data processing system being configured to:
calculate a baseline pressure from the data;
detect sucks by comparing pressure fluctuations to known characteristics;
detect swallows by comparing pressure fluctuations to known characteristics;
detect respiration by comparing temperature fluctuations caused by inhalation and exhalation by the infant to known characteristics;
calculate a second time period between the sucks and the swallows to detect bursts;
calculate a third time period without the sucks and without the swallows;
calculate at least one biomarker for the infant or the feeding instance;
analyze the at least one biomarker to track maturation and neuro-development of the infant; and
transfer results of the feeding instance to at least one of an engine executable on a computing device, a cloud, a graphical user interface (GUI) of the computing device, or a telehealth interface of the computing device, wherein the results of the feeding instance comprise curves depicting at least one of feeding trace data, maturation progress data, duration of quality data, and recovery data.

49. The device of claim 48, wherein the computerized data processing system comprises one or more algorithms that are configured to: smooth the data and separate the sucks from the swallows, select single and multiple suck signals in a suck/swallow sequence, and detect a correlation between the suck/swallow sequence and respirations, and/or wherein, subsequent to establishing the baseline pressure from the data, the computerized data processing system is further configured to:
determine an amplitude of suck, swallow and respiration curves; and
detect a difference in a shape of a suck curve as compared to a swallow curve; and/or
wherein the baseline pressure is calculated from the data by taking a measurement at a beginning and an end of the feeding instance or by taking a measurement at the beginning and at the end of every burst during the feeding instance.

50. The device of claim 48, wherein the device further comprises a pyroelectric detector or a pyroelectric film, and wherein the pyroelectric detector or the pyroelectric film is further configured to collect a respiration measurement and/or wherein the computerized data processing system is further configured to:
analyze the respiration measurement;
compare the respiration measurement to measurements taken during the feeding instance when the infant is engaging in sucking and swallowing actions; and
determine a frequency or an absence during the sucking and swallowing actions, a quality of a shape, and a comparison of the frequency and a strength during a burst and during a rest period.

51. The device of claim 48, wherein the at least one biomarker is associated with oral cavity pressure changes or respiratory changes by temperature variations, and/or wherein each sensor of the at least one sensor is selected from the group consisting of: a pressure sensor and a respiratory sensor.

52. The device of claim 51, wherein the at least one sensor comprises a respiratory sensor, wherein the respiratory sensor is located between the embedded electronic components and a personal computer connection, and wherein the respiratory sensor is configured to measure changes in temperature in exhalation.

53. The device of claim 51, wherein the at least one sensor comprises a pressure sensor, wherein the pressure sensor is located inside of the bottle, and wherein the pressure sensor is configured to measure an oral cavity pressure of the infant.

54. The device of claim 48, wherein the computerized data processing system is further configured to differentiate each of the sucks as a nutritive suck or a non-nutritive suck and/or to assess a ratio of suck to swallow in both count and strength, clarity of signal, and coordination between suck/swallow and respiration.
